(19) United States
(12) Patent Application Publication
(10) Pub. No.: US 2007/0010428 A1
(43) Pub. Date: Jan. 11, 2007

(54) Inhibitors of signal transduction and activator of transcription 3

(76) Inventors: John S. McMurray, Houston, TX (US); Zhiyong Ren, Houston, TX (US); David R. Coleman IV, Houston, TX (US); Pijus K. Mandal, Houston, TX (US); Xiaomin Chen, Houston, TX (US); Warren Liao, Houston, TX (US)

Correspondence Address: VINSON & ELKINS, L.L.P., 1001 FANNIN STREET, 2300 FIRST CITY TOWER, HOUSTON, TX, 77002-6760, US

(21) Appl. No.: 11/219,530

(22) Filed: Sep. 2, 2005

Related U.S. Application Data
(60) Provisional application No. 60/607,317, filed on Sep. 3, 2004.

(51) Int. Cl. A61K 38/08; A61K 38/06; A61K 38/05

(52) U.S. Cl. 514/7; 514/17; 514/18

(57) ABSTRACT

Stat3 inhibitor compounds are disclosed, wherein the compounds are structural analogs of Ac-pTyr-Leu-Pro-Gln-Thr-NH₂ and bind to the SH2 domain of Stat3 under physiological conditions to inhibit a cellular signaling activity of Stat3.

### CROSS REFERENCE TO RELATED APPLICATIONS

This Application claims benefit of priority under 35 USC 119(e) to U.S. Provisional Application No. 60/607,317, filed Sep. 3, 2004, the disclosure of which is incorporated herein in its entirety by reference.

### STATEMENT REGARDING FEDERALLY SPONSORED RESEARCH OR DEVELOPMENT

The U.S. Government may have certain rights in this invention because the work performed during development of this disclosure was supported at least in part by NIH grant #CA 96652.

### BACKGROUND OF THE INVENTION

Signal transducer and activators of transcription 3 (Stat3) is a member of the STAT family of transcription factors that relate signals from extracellular signaling protein receptors on the plasma membrane directly to the nucleus (reviewed in Stark et al, 1998, Bromberg & Darnell, 2000; Levy and Darnell, 2002). Stat3 was discovered as a major component in the acute phase response to inflammation (Akira et al., 1994) and as a key mediator of interleukin 6 (IL-6) (Zhong 1994a) and epidermal growth factor signaling (Zhong 1994b). Like all STATS, Stat3 is composed of an amino-terminal oligomerization domain, a coiled coil domain, a DNA binding domain, a linker domain, a Src homology 2 (SH2) domain, and a C-terminal transactivation domain (FIG. 1).

In IL-6 signaling, on binding of the cytokine to its receptor, JAK kinases are recruited to the co-receptor, gp130, which becomes phosphorylated on several tyrosine residues (Stahl et al, 1995, Gerhartz et al, 1996) (FIG. 2). Stat3, via its SH2 domain, binds to the phosphotyrosine residues on gp130 and is then phosphorylated on Tyr705, a conserved tyrosine just C-terminal to the SH2 domain, by JAK2. Upon phosphorylation, termed activation, Stat3 forms homodimers and/or heterodimers with Stat1 via reciprocal interactions between the SH2 domains and the phosphotyrosine residue. The dimers then translocate to the nucleus and bind specific DNA sequences where they, in cooperation with other transcription factors, regulate gene expression (Bromberg and Darnell, 2000; Levy and Darnell, 2002, Stark et al, 1998).

Downstream targets of Stat3 include Bc1-X_{L}, a member of the bc1-2 family of anti-apoptotic proteins, cell cycle regulators such as cyclin D1 and p21^{WAF1/CIP1} and other transcription factors including c-myc and c-fos. In EGF signaling, Stat3 has been reported to bind directly to phosphotyrosine residues on the EGFR and to be activated by the kinase activity of the receptor (Zhong et al., 1994b; Coffer & Kruijer, 1995; Zhang et al, 2003). Further studies imply that Src kinases first bind to EGFR via their SH2 domains and recruit Stat3 vis SH3 domain interactions with polyproline helices (Schreiner et al 2002).

Stat3 transmits signals from other IL-6-type cytokines that utilize gp130 such as ciliary neurotrophic factor, leukemia inhibitory factor, oncostatin M, IL-10 (Weber-Norte et al 1996a), and granulocyte colony-stimulating factor (Chakraborty et al, 1999). In addition to cytokines, it has also been shown to be involved signaling from the epidermal growth factor (Zhong, 1994), platelet derived growth factor, and vascular endothelial growth factor (Niu et al 2002).

It would be useful, therefore to identify compounds that bind to the SH2 domain, and are effective to inhibit Stat3 binding to receptor,and that also inhibit dimer formation, subsequent translocation to the nucleus, DNA binding, and transcription.

Stat3 has been shown to be constitutively activated in cancers of the head and neck (reviewed in Song and Grandis 2000), breast (Garcia et al, 1997), brain (Schaefer et al, 2002), prostate (Dhir et al, 2002), lung (Seki et al, 2004), ovary (Huang et al, 2000), pancreas (Sholz et al, 2003), leukemia (reviewed in Benekli et al, 2003) multiple myeloma, lymphoma (Weber-Norte et al, 1996a) and others (reviewed in Bowman et al, 2000; Buettner et al, 2002; Bromberg, 2002; Darnell, 2002; Yu and Jove 2004). As mentioned, Stat3 up-regulates the anti-apoptotic gene Bc1-X_{L} and the cell cyclic gene cyclin D1, thereby promoting cell survival and cell cycle progression. It has also been shown to up-regulate VEGF expression and thus has a potential role in angiogenesis (Niu et al, 2002a). Inhibition of Stat3 activity by the introduction of antisense oligonucleotides or dominant negative constructs has been shown to induce apoptosis and reduce cell growth, and soft agar colony formation in several tumor cell lines exhibiting constitutively active Stat3 (Burke et al, 2001 Catlett-Falcone et al. 1999; Niu et al; 1999; Grandis et al, 2000, Grandis et al, 1998). Delivery of an oligonucleotide decoy, i.e. a 15-mer double stranded section of the Stat3 response element, also deactivated Stat3 resulting in apoptosis on head and neck squamous cells (Leong et al., 2003). In those cells driven by EGF signaling, introduction of small molecule EGFR inhibitors reduced Stat3 activation and resulted in cell death (Real et al 2002). A small molecule inhibitor of the Src kinase also inhibits Stat3 activation, induces apoptosis and inhibits cell growth in a breast cancer cell line (Garcia et al Oncogene 2001). These studies all demonstrate that inhibiting Stat3 activity decreases growth and induces cell death in a variety of cell lines and therefore validate Stat3 as an attractive target for anti-cancer drug design. (reviewed in Bowman et al, 2000; Darnell, 2002, Buettner et al, 2002, Bromberg, 2002, Yu et al., 2004)

Several inhibitors of Stat3 have been described, including small molecule inhibitors, oligonucleotides, and peptide and peptide-based inhibitors. An example of a small molecule, non-peptide inhibitor of Stat3 is Curcurbitacin 1 (FIG. 3), discovered by Blaskovich et al. (2003) in screening the NCI Diversity Set for compounds inhibiting phosphorylation of Stat3. Curcurbitacin 1 is a natural product member of the cucurbitacin family of compounds that are isolated from various plant families such as the Cucurbitaceae and Cruciferae and have been used as folk medicines for centuries in countries such as China and India (Blaskovich et al., 2003). This natural product inhibits the phosphorylation of Stat3, and the translation of a Stat3-dependant reporter gene. It also inhibits the growth of Stat3 dependant cell lines in culture and in xenograft models. No evidence of its directly binding to Stat3 was given in the Blaskovitch paper. (Patent: Sebti et al et al, 2002)

Several reports in the literature have described the use of antisense oligonucleotides to inhibit Stat3 expression and the use of oligonucleotides to express dominant-negative Stat3 to study the effect of reduced Stat3 activity on cell proliferation (Burke et al, 2001 Catlett-Falcone et al. 1999; Niu et al; 1999; Grandis et al, 2000, Grandis et al, 1998). Antisense oligonucleotides have been patented by researchers at the Moffat Cancer Center in Florida (Yu et al, 2002). An antisense oligonucleotide is reportedly being developed by Isis Pharmaceuticals (Karras 2000a, Karras et al 2000b).

Turkson et al, 2001, reported the use of tri-, tetra-, and penta-peptide inhibitors of Stat3 dimerization and DNA binding (See also the patent Jove et al., 2000). These peptides were based on the sequence surrounding Tyr⁷⁰⁵, the phosphorylation site of Stat3 (Pro-pTyr⁷⁰⁵-Leu-Lys-Thr-Lys-Phe, SEQ ID NO:1) and were reported to have IC₅₀ values of 200-400 μM using EMSA. Since IC₅₀ values can vary depending on experimental conditions the present inventors tested a similar peptide, Ac-pTyr-Leu-Lys-Thr-Lys-Phe-NH₂, SEQ ID NO:2, in their own laboratory, using EMSA, and found the IC₅₀ value was 20 μM (Peptide 2, Table 1). In contrast, the lead compound of the present disclosure, Ac-pTyr-Leu-Pro-Gln-Thr-Val, SEQ ID NO:3, had an IC₅₀ value of 0.15 μ.M, a >100-fold increase in potency (Peptide 1, Table 1). Since peptides based on the phosphorylation site of Stat3, the basis of the Turkson et al. peptides, are very low in affinity, there is a need in the art for compounds having the advantage of higher potency.

In addition to the small peptides, Turkson et al. described a phosphorylated 18-residue peptide, H-Pro-pTyr-Leu-Lys-Thr-Lys-Ala-Ala-Val-Leu-Leu-Pro-Val-Leu-Leu-Ala-Ala-Pro-OH, SEQ ID NO:4. This peptide contains the sequence from Stat3 Tyr⁷⁰⁵ (H-Pro-pTyr-Leu-Lys-Thr-Lys, SEQ ID NO:5) fused to the 12-residue membrane transporter sequence (mts) described below. This peptide was shown to have activity in cell culture but very high concentrations (0.5-1 mM) were required to inhibit luciferase reporter gene expression, Stat3 nuclear translocation, and cell growth. There is a need therefore for peptidomimetic molecules that are active at lower concentrations.

TABLE 1

The inhibition of Stat3 dimerization and DNA
binding by receptor or Stat3-derived phospho-
peptides as measured by EMSAs (from Ren et
al, 2003).

| | | | | | |
|---|---|---|---|---|---|
| | | | | SEQ | |
| | Receptor/ | Tyr | | ID | IC₅₀ |
| Peptide | Protein | Position | Sequence^{a} | NO | (μM)^{b} |

| | | | | | |
|---|---|---|---|---|---|
| 1 | gp130 | 904 | Y(p)LPQTV | 3 | 0.15 |
| 2 | Stat3 | 705 | Y(p)LKTKF | 2 | 20 |
| 3 | EGFR | 1068 | Y(p)INQSV | 6 | 30 |
| 4 | EGFR | 1086 | Y(p)HNQPL | 7 | 150 |

^{a}Alt peptides are acetylated on the N-terminus and are C-terminal amides
^{b}IC₅₀ values were determined by EMSA and are the averages of two determinations

A further publication by Turkson et al. (2004) includes a series of tripeptide mimetics of the structure R₁-pTyr-Leu. The structures are shown in FIG. 4. This publication does not indicate if the C-termini are amides (R₂═NH₂) or carboxyl groups (R₂═OH). R₁ is a set of aromatic rings with varying substitution. The series exhibits a broad range of IC₅₀ values as determined by EMSA. The three highest affinity compounds are rather low potency. ISS 610 was tested in cell culture models and it required 1 mM concentrations for activity in luciferase reporter and cell growth assays, still much higher than needed.

D. Tweardy and colleagues (Shao et al. 2003) reported that peptides surrounding tyrosines 1068 and 1086 of the EGF receptor, when appended to the same mts peptide, abrogate Stat3 dimerization and DNA binding in cell nuclear extracts and in cell cultures. The two peptides are Leu-Pro-Val-Glu-pTyr-lle-Asn-Gln-Ser-mts, SEQ ID NO:8 (Y1068-mts) and Val-Gln-Asn-Pro-Val-pTyr-His-Asn-Gln-Pro-Leu-Asn-mts, SEQ ID NO:9 (Y1086-mts). Although the inventors have not tested these peptides, hexapeptides from EGFR 1068 (peptide 3, Table 1) and 1086 (peptide 4, Table 1) have been tested for inhibition of dimer formation and DNA binding ability using EMSA and IC₅₀ values of 30 and 150 μM (Ren et al, 2003) were found. There is still a need, therefore for compounds of greater potency, preferably with IC₅₀ values below 1.0 μM.

### SUMMARY

The present disclosure may be described in certain embodiments as a set of compounds that bind to the SH2 domain of Stat3 and inhibit the signaling functions of Stat3, such as the ability of Stat3 (i) to bind to phosphotyrosine residues on the receptors of cytokines or growth factors, (ii) to form dimers that translocate to the nucleus and (iii), to bind (in dimeric form) to specific DNA sequences and initiate transcription of antiapoptotic genes (e.g., Bc1-x_{L}), cell cycle genes (e.g. cyclin D1, p21^{WAF1/CIP1}, and others. Disclosed compounds include peptidomimetics derived from a lead phosphopeptide targeted to the SH2 domain of Stat3: Ac-pTyr-Leu-Pro-Gln-Thr-Val-NH₂, SEQ ID NO:3 (peptide 1), which was discovered by the present inventors to be a high affinity inhibitor of Stat3 dimerization and DNA binding in vitro (Ren et al., 2003) (Table 1). The amino acid sequence of the lead peptide is residues 904-909 of gp130, a component of the IL-6 receptor. A series of small molecule peptidomimetics (compounds 5-19) is listed in FIG. 14 along with IC₅₀ values from an in vitro fluorescence polarization assay.

An aspect of the disclosure is also a peptide having the following sequence:

F2PmCinn-Leu-Pro-Gln-Thr-Val-Ala-Ala-Val-Leu-Leu-Pro-Val-Leu-Leu-Ala-Ala-Pro-NH₂, SEQ ID NO:10 ( Peptide 21), which is a modified version of the lead peptide appended to the mts (membrane transporter sequence). In preparing peptide 21, the phosphotyrosine of the lead peptide was replaced by 4-phosphonodifluoromethylcinnamide (F2PmCinn, FIG. 6). 4-Phosphosphoryloxycinnamate was found in the peptidomimetic structure activity relationship (SAR) studies in the development of peptidomimetic inhibitors described below. The phosphonodifluoromethyl group is a phosphate isostere that is not hydrolysable by phosphatases (Wrobel and Dietrich, 1993). The cinnamide unit is a non-rotatable and non-amino acid tyrosine mimic. It imparts stability to degradation by proteases. The hydrophobic, 12-residue mts (Ala-Ala-Val-Leu-Leu-Pro-Val-Leu-Leu-Ala-Ala-Pro-NH₂, SEQ ID NO:11), derived from the h-region of the signal sequence of Kaposi fibroblast growth factor (Rojas et al, 1998) is capable of delivering hydrophobic “cargo” across cell membranes, and has been used to deliver other, low affinity Stat3 inhibitors by other researchers (Turkson et al, 2001; Shao et al, 2003).

Peptide 21 and a pro-drug form of one of the disclosed peptidomimetics, F2Pm(POM)Cinn-Haic-Gln-NHBn (23) are shown herein to inhibit the migration of Stat3 to the nucleus as well as the expression of a luciferase reporter gene possessing a Stat3 sensitive promoter (POM=pivaloyloxymethyl, Sastry et al, 1992). Therefore, these compounds are useful reagents for the study of Stat3 physiology and its role in cancer biology, and the disclosed small molecule peptidomimetics may further have activity as chemotherapeutic agents for Stat3-sensitive tumors. Leu-Ala-Ala-Pro-OH, An aspect of the disclosure, therefore, is a composition comprising a Stat3 inhibitor compound, wherein the compound comprises a structural analog of Ac-pTyr-Leu-Pro-Gln-Thr-NH₂, SEQ ID NO:12 in which one or more amino acids have been replaced with a structural analog of the amino acid or amino acids, wherein the compound binds to the SH2 domain of Stat3 under physiological conditions and wherein the binding of the compound inhibits a cellular signaling activity of Stat3. For example, preferred analogs include those in which the structure of important peptide-protein contacts within the lead peptide are maintained, e.g. pY+1 backbone NH and the pY+3 Gln side chain NH₂ protons and the fact that the Leu-Pro peptide bond is trans. pY+1 and pY+3 indicate the 1st and 3rd amino acids, respectively, to the right, or toward the C terminus of the phosphor-tyrosine in the lead peptide amino acid sequence.

Preferred compositions thus include those in which the Ac-pTyr has been replaced with a more stabile structural analog. Preferred analogs include this in which the Ac-pTyr has been replaced with 4-phosphonodifluoromethylcinnamide (F2PmCinn), including those in which pivaloyloxymethyl is added to one or more of the phosphonyl oxygen atoms of the 4-phosphonodifluoromethylcinnamide, those in which Ac-pTyr has been replaced with 3-phosphoryloxyindole-2-carboxylate, those in which Ac-pTyr has been replaced with 3-phosphonodifluoromethylindole-2-carboxylate, and those in which pivaloyloxymethyl is added to one or more of the phosophonyl oxygen atoms of the -phosphonodifluoromethylindole-2-carboxylate. Further preferred compositions include those in which Ac-pTyr has been replaced with 4-phosphoryloxycinnamide.

Certain of the disclosed structural analogs include those in which the Leu or Leu-Pro have been replaced with structural analogs. For example, Leu may be replaced with cyclohexylalanine, Leu-Pro may be replaced with 5-(amino)-1,2,4,5,6,7-hexahydro-4-oxo-(2S,5S)-azepino[3,2,1-hi]indole-2-carboxylic acid (Haic), or (3S,6S,9S) 2-oxo-3-amino-1-azabicyclo[4.3.0]nonane-9-carboxylic acid (ABN), or Pro may be replaced with 3,4-methanoproline. Disclosed structural analogs also include those in which Gln has been replaced with pyrrolidinoacetamide. The present inventors have also discovered that the Thr-NH2 can be replaced by a more hydrophobic group and in particular by a benzene ring structure.

It is an aspect of the invention that, while single amino acids may be replaced with structural analogs, there are certain advantages offered by replacing two or more amino acids, or even all the amino acids simultaneously. As such, certain preferred embodiments of the disclosed compounds include compositions containing compounds based on the lead peptide (peptide 1) in which Ac-pTyr has been replaced with 4-phosphoryloxycinnamide, and the Thr-NH₂ has been replaced with a benzyl group; those in which the Ac-pTyr has been replaced with 4-phosphonodifluoromethylcinnamide, the Leu has been replaced with cyclohexylalanine, and the Thr-NH₂ has been replaced with a benzyl group; those in which Ac-pTyr has been replaced with 3-phosphoryloxyindole-2-carboxylate, the Leu has been replaced with cyclohexylalanine, and the Thr-NH₂ has been replaced with a benzyl group; those in which Ac-pTyr has been replaced with 4-phosphoryloxycinnamide, the Leu-Pro has been replaced with 5-(amino)-1,2,4,5,6,7-hexahydro-4-oxo-(2S,5S)-azepino[3,2,1-hi]indole-2-carboxylic acid, and the Thr-NH₂ has been replaced with a benzyl group; those in which the Ac-pTyr has been replaced with 4-phosphonodifluoromethylcinnamide, the Leu-Pro has been replaced with 5-(amino)-1,2,4,5,6,7-hexahydro-4-oxo-(2S,5S)-azepino[3,2,1-hi]indole-2-carboxylic acid and the Thr-NH₂ has been replaced with a benzyl group; those in which Ac-pTyr has been replaced with 3-phosphoryloxyindole-2-carboxylate, the Leu-Pro has been replaced with 5-(amino)-1,2,4,5,6,7-hexahydro-4-oxo-(2S,5S)-azepino[3,2,1-hi]indole-2-carboxylic acid, and the Thr-NH₂ has been replaced with a benzyl group; those in which Ac-pTyr has been replaced with 4-phosphoryloxycinnamide, the Leu-Pro has been replaced with (3S,6S,9S) 2-oxo-3-amino-i-azabicyclo[4.3.0]nonane-9-carboxylic acid, and the Thr-NH₂ has been replaced with a benzyl group; and those in which Ac-pTyr has been replaced with 4-phosphoryloxycinnamide, the Leu has been replaced with cyclohexylalanine, the Pro has been replaced with 3,4-methanoproline, and the Thr-NH₂ has been replaced with a benzyl group.

Further preferred embodiments include those compositions based on the lead peptide (peptide 1), in which Ac-pTyr has been replaced with 4-phosphonodifluoromethylcinnamide, the Leu has been replaced with cyclohexylalanine, the Pro has been replaced with 3,4-methanoproline, and the Thr-NH₂ has been replaced with a benzyl group; those in which Ac-pTyr has been replaced with 3-phosphoryloxyindole-2-carboxylate, the Leu has been replaced with cyclohexylalanine, the Pro has been replaced with 3,4-methanoproline, and the Thr-NH₂ has been replaced with a benzyl group; those in which Ac-pTyr has been replaced with 4-phosphoryloxycinnamide, the Leu-Pro has been replaced with 5-(amino)-1,2,4,5,6,7-hexahydro-4-oxo-(2S,5S)-azepino[3,2,1-hi]indole-2-carboxylic acid, and the Gln has been replaced with pyrrolidinoacetamide; those in which Ac-pTyr has been replaced with 4-phosphonodifluoromethylcinnamide, the Leu-Pro has been replaced with 5-(amino)-1,2,4,5,6,7-hexahydro-4-oxo-(2S,5S)-azepino[3,2, 1-hi]indole-2-carboxylic acid, and the Gin has been replaced with pyrrolidinoacetamide; or those in which Ac-pTyr has been replaced with 4-phosphonodifluoromethylcinnamide, wherein pivaloyloxymethyl is added to one of the phosphonyl oxygen atoms of 4-phosphonodifluoromethylcinnamide, the Leu-Pro has been replaced with 5-(amino)-1,2,4,5,6,7-hexahydro-4-oxo-(2S,5S)-azepino[3,2,1-hi]indole-2-carboxylic acid, and the Thr-NH₂ has been replaced with a benzyl group.

It is an aspect of the present disclosure that the preferred compounds may also include molecules to impart more hydrophobic characteristics to the compound, particularly at the N-terminus, or the analog thereof. In certain embodiments, then a benzene or other hydrophobic ring structure may be added at that position, or one could add or attach a membrane transporter sequence to any of the disclosed compounds. A preferred membrane transporter sequence is a peptide with the following amino acid sequence: Ala-Ala-Val-Leu-Leu-Pro-Val-Leu-Leu-Ala-Ala-Pro-NH₂, SEQ ID NO: 11.

It is a further aspect of the disclosure that the described compositions may include any the described compounds dissolved or suspended in a pharmaceutically acceptable carrier. The phrases “pharmaceutically and/or pharmacologically acceptable” refer to molecular entities and/or compositions that do not produce an adverse, allergic and/or other untoward reaction when administered to an animal. As used herein, “pharmaceutically acceptable carrier” includes any and/or all solvents, dispersion media, coatings, antibacterial and/or antifungal agents, isotonic and/or absorption delaying agents and/or the like. The use of such media and/or agents for pharmaceutical active substances is well known in the art. Except insofar as any conventional media and/or agent is incompatible with the active ingredient, its use in the therapeutic compositions is contemplated.

An additional preferred embodiment is a composition comprising a compound having the structure: F2PmCinn-Leu-Pro-Gln-Thr-Val-Ala-Ala-Val-Leu-Leu-Pro-Val-Leu-Leu-Ala-Ala-Pro-NH₂, SEQ ID NO:10.

In certain aspects the disclosure may also be described as a method of inhibiting the signaling activity of Stat3 in a cell, cell culture or organism, the method including contacting the cell with a compound that binds to the SH2 domain of Stat3, wherein the molecule includes a structural analog of phosphorylated Tyr 904 of gp130, and more particularly may be described as such a method in which the compound includes a structural analog of Ac-pTyr-Leu-Pro-Gln-Thr-NH₂, SEQ ID NO:12 in which one or more amino acids have been replaced with a structural analog of the replaced amino acids. In practicing preferred embodiments of the described methods, the compound binds to the SH2 domain and inhibits Stat3 dimerization, and/or it may inhibit translocation of Stat3 to the nucleus of the cell, and/or inhibit activation of transcription of Stat3 responsive genes in the cell.

Throughout this disclosure, unless the context dictates otherwise, the word “comprise” or variations such as “comprises” or “comprising,” is understood to mean “includes, but is not limited to” such that other elements that are not explicitly mentioned may also be included. Further, unless the context dictates otherwise, use of the term “a” may mean a singular object or element, or it may mean a plurality, or one or more of such objects or elements.

Certain aspects of the disclosure also involve synthesis of Azabicyclo[X.Y.0]-alkane aminoacids (AZABIC). Azabicyclo[X.Y.0]-alkane aminoacids are conformationally rigid dipeptide mimics that constrain three backbone dihedral angles within a fused bicyclic framework (Hanessian, S.; McNaughton-Smith, G.; Lombart, H.-G.; Lubell, W. D. Tetrahedron 1997, 53, 12789. (b) Gillespie, P.; Cicariello, J.; Olson, G. L. Biopolymers, 1997, 43, 191; (c) Eguchi, M.; Kahn, M. Mini Reviews in Medicinal Chemistry, 2002, 2, 447). The growing use of these dipeptide units in structure-activity relationship studies of biologically active peptides has created a demand for new, efficient methodology for their synthesis. The present disclosure may also include employing AZABIC mimetics in SAR studies of peptide-based inhibitors of oncogenic signal transduction proteins such as Stat3. A preferred and efficient synthesis of 3-(Fmoc-amino)-azabicyclo[4.3.0]-nonane-2-carboxylate (n=1) and its homologue 3-(Fmoc-amino)-azabicyclo[5.3.0]-decane-2-carboxylate (n=2) are disclosed herein. Boc-pyroglutamate or Boc-homopyroglutamate is cleaved with a vinyl Grignard reagent to produce acyclic γ or δ-vinyl ketones. Michael addition of N-diphenylmethylene glycine tert-butyl ester to the vinyl group produces diamino dicarboxylate precursors, which, on hydrogenolysis, undergo double cyclization to give the fused bicylic ring system. Acidolysis of the tert-butyl-based protecting groups followed by treatment with Fmoc-OSu results in Fmoc-protected dipeptide mimetics ready for solid phase synthesis.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a schematic representation of the structure of a Stat3 protein.

FIG. 2 is a schematic representation of Stat3 activation.

FIG. 3 is the molecular structure of Curcurbitacin 1.

FIG. 4 shows the general structure of Stat3 peptidomimetics and the three highest affinity compounds from Turkson et al. (2004).

FIG. 5 is FP curves of Stat3. Left panel, Titration of full length Stat3 into 10 nM solutions of FP probe. Each protein concentration was run in duplicate and the mean ±½ the difference between the high and low value was plotted. Right panel, a competition curve in which increasing concentrations of peptide 1 were added to solutions of 10 nM probe and 40 nM Stat3 (final concentrations). Each peptide concentration was run in duplicate and the mean ±½ the difference between the high and low value was plotted. A Perkin Elmer (formerly Packard) 204DT Multiprobe liquid handling robot was used to prepare serial dilutions of the inhibitors and to add these solutions to Stat3-probe solutions. The dilutions and additions were done in 96-well plates and FP was read on a Tecan Polarian plate reader. By adding inhibitors to solutions of FP probe and Stat3, competition curves were generated and IC₅₀ values were obtained and are reported in FIG. 14. FIG. 5, right panel shows an example of a competition curve of peptide 1 to determine the IC₅₀.

FIG. 6 is the molecular structures of phosphotyrosine and preferred embodiments of mimetics. PTyr=phosphotyrosine; pCinn=phosphoryloxycinnamide, p1nd=5-phosphoryloxyindole-2-carboxamide, F2Pmp=4-phosphonodifluoromethylphenylalanine; F2PmCinn=4-phosphonodifluoromethylcinnamide.

FIG. 7 shows the structure of residues 702-711 of Stat3 bound to the SH2 domain. Depicted is CO—Cα-Cβ-Cγ (arom) dihedral angle of phosphotyrosine 705 (From Becker et al., 1998).

FIG. 8 is the structure of Compound 23, pro-drug F2PmCinn(POM)-Haic-Gln-NHBn.

FIG. 9 is a bar graph of data showing inhibition of expression of the luciferase reporter by mts peptides in HEPG2 cells. Cells were treated with 50 or 100 μM of peptides 21, 24, or 25.

FIG. 10 is a bar graph of data showing inhibition of expression of the luciferase reporter by mts peptides in HEP3B cells with pro-drug 23.

FIG. 11 is an image of a gel showing EMSA of nuclear extracts of HEPG2 cells inhibited with peptides. Shown are gels from two independent experiments. Lane 1, control: no IL-6 or inhibitor. Lane 2-6 are all stimulated with IL-6, 6 ng/mL. Lane 2, no inhibitor, Lane 3 phosphopeptide 24, Lane 4, control peptide 25, Lane 5, F2PmCinn peptide 21, Lane 6, prodrug 23.

FIG. 12 shows the structures of the three main scaffolds for peptidomimetic design, derived from the Leu Pro dipeptide central unit of peptide 1.

FIG. 13 is a preferred synthetic scheme for synthesis of 4-phosphondifluoromethyl)-cinnamic acid.

FIGS. 14A-E is a series of small molecule peptidomimetics (compounds 5-19) along with IC₅₀ values from an in vitro fluorescence polarization assay.

FIG. 15 shows the structure of a compound in which amino acids from the lead peptide have been replaced with structural analogs, and in particular, in which the Ac-pTyr has been replaced with 4-phosphoryloxycinnamide, the Leu-Pro has been replaced with (3S,6S,9S) 2-oxo-3-amino-1-azabicyclo[4.3.0]nonane-9-carboxylic acid, and the Thr-NH₂ has been replaced with a benzyl group. This compound was found to have an IC₅₀ of 400 nM. The second structure is the same compound in which the tyrosine along has been changed to a difluoromethyl phosphonate and the third structure includes the attachment of the pivaloyloxymethyl prodrug groups to the phosphonate oxygens.

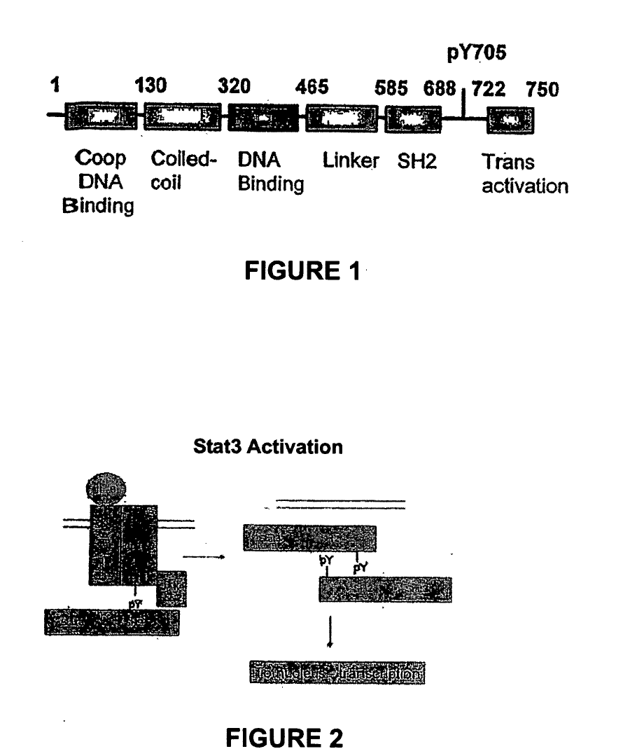

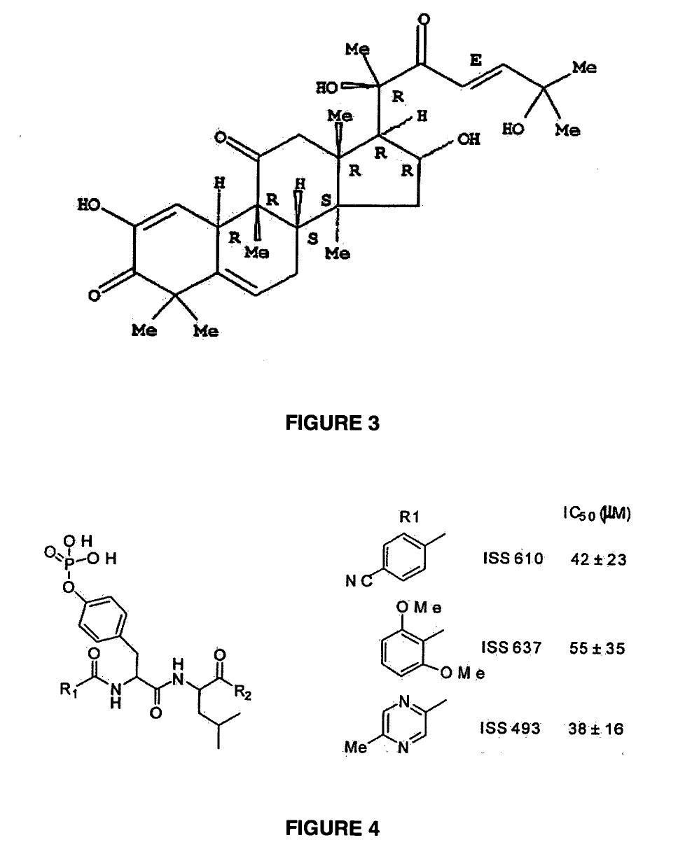

### DETAILED DESCRIPTION

An aspect of the present disclosure is a set of compounds that inhibit Stat3 activity by y binding to its SH2 domain. A series of peptidomimetics is disclosed herein that binds to the Stat3 SH2 domain in in vitro fluorescence polarization assays. A modified phosphopeptide and a small molecule prodrug inhibit State3 dimerization, translocation to the nucleus and subsequent transcription of Stat3-activated luciferase reporter genes in model cell lines HEPG2 and HEP3B, thus demonstrating the potential of these compounds to serve as reagents for the study of Stat3 activity and, in the case of the small molecule, chemotherapeutic agents for Stat3 responsive tumors.

Development of Peptidomimetic Inhibitors of Stat3

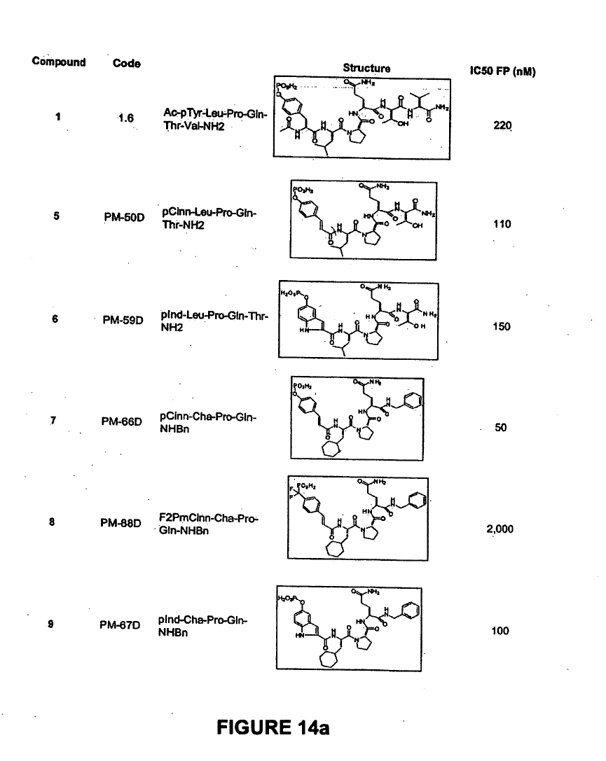

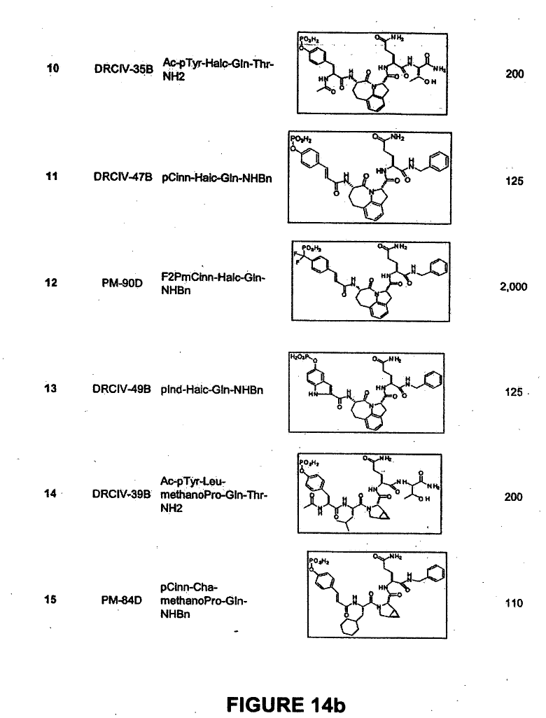

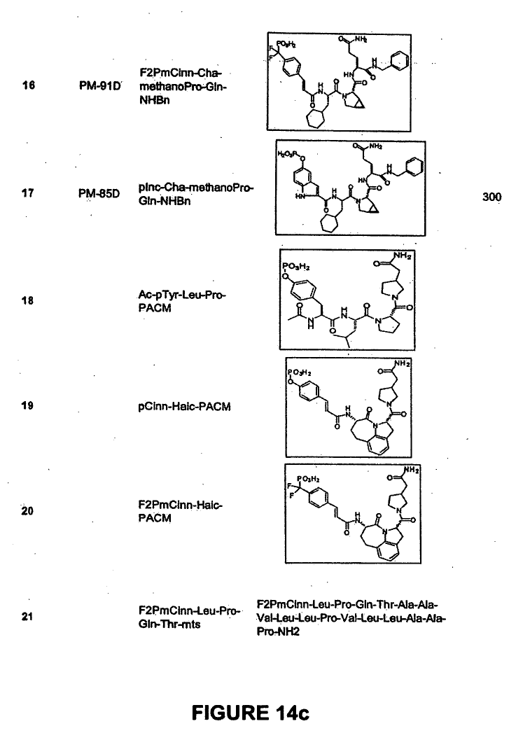

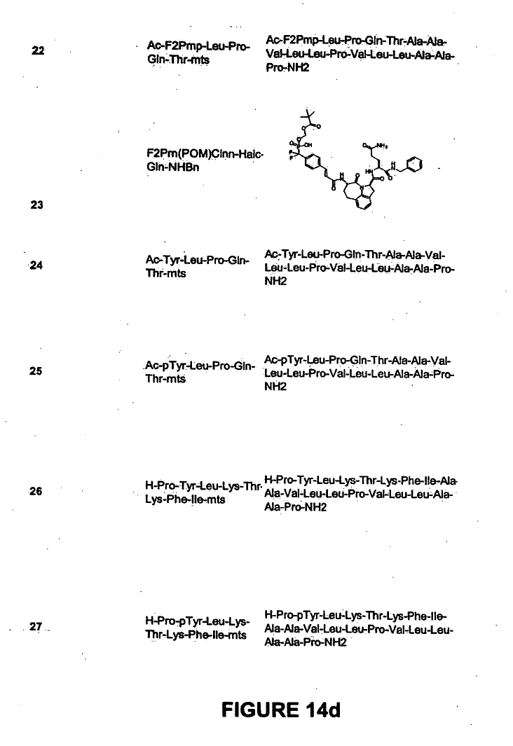

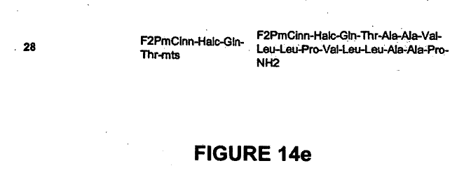

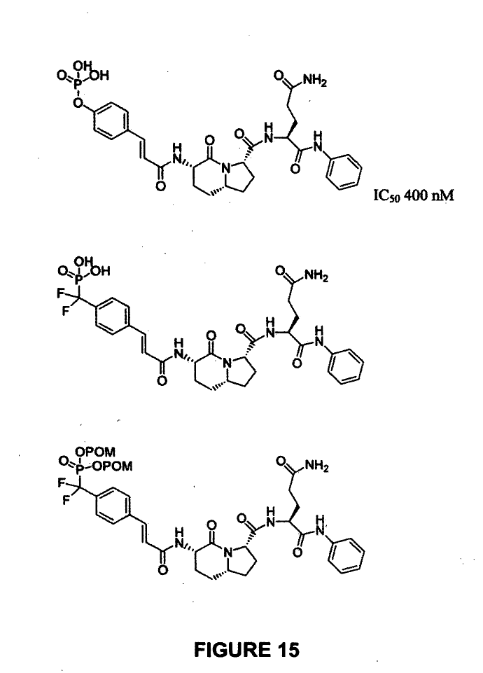

To find a lead compound for inhibitor development, a series of phospho-hexapeptides derived from known receptor docking sites for Stat3 were synthesized and assayed for their ability to inhibit Stat3 dimerization and DNA binding using electrophoretic mobility shift assays (EMSA) (Ren et al., 2003). Of this preliminary series, the most potent was peptide 1 (Ren et al., 2003). It was discovered that the Val at position pY+5 could be eliminated and thus the pentapeptide was used as the template. SAR experiments were conducted systematically replacing each amino acid. The goal was to find non-peptidic groups that would be stable to phosphatases and proteases, and that would be more hydrophobic to allow greater bioavailability. Several novel high affinity structures were discovered, for example (compounds 5-19, FIG. 14). The small peptidomimetics were assayed for their ability to bind to the SH2 domain of Stat3 using fluoresence polarization.

Fluorescence polarization (FP) is a rapid and easy method for the measurement of peptide-protein interactions, drug-protein interactions, and drug-oligonucleotide interactions, and is readily adaptable to high throughput formats (reviewed in Nasir & Joley, 1999; Owicki, 2000). FP involves exciting a fluorophore with polarized light and taking the ratio of fluorescence at right angles after a brief period of time. Small molecules rotate in solution more rapidly than do macromolecules. When the FP probe is free the degree of polarization is smaller than when the molecule is bound to Stat3. A fluorescein-labeled version of peptide 1 (fluorescein-5-carboxyl-Ala-pTyr-Leu-Pro-Gln-Thr-Val-NH₂, SEQ ID NO:13), called the FP probe, was synthesized for use in fluorescence polarization (FP) assays. A binding curve was generated by titrating full length Stat3 into solutions of the FP probe (FIG. 5). The FP probe has a Kd of ca. 50 nM for binding to full length Stat3.

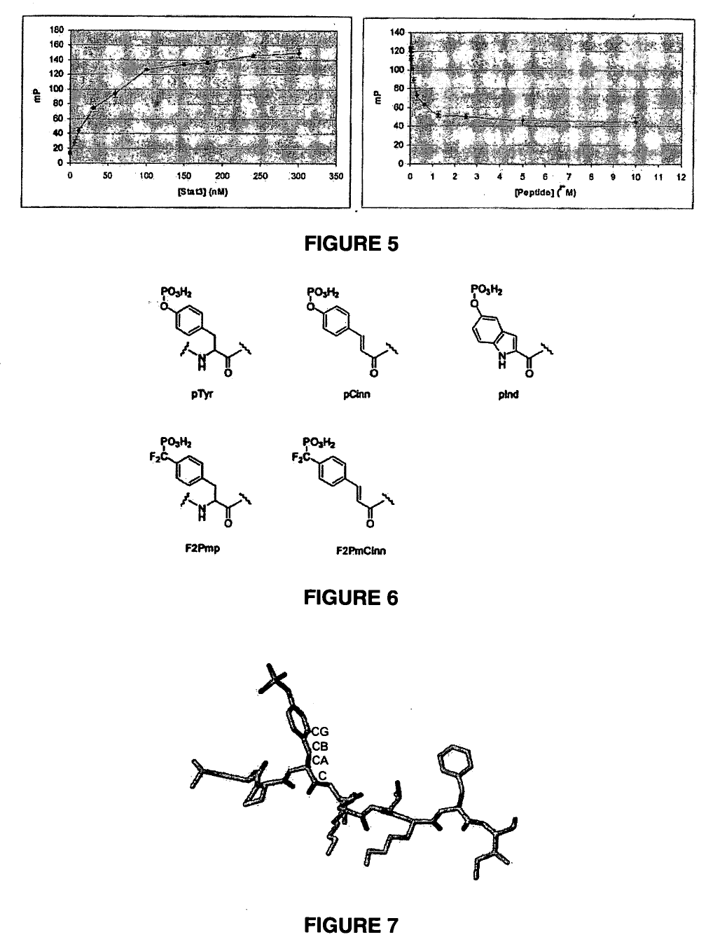

The phosphotyrosine was replaced with 4-phosphoryloxycinnamide and 3-phosphoryloxyindole-2-carboxylate groups (compounds 5 and 6). These are non-amino acid mimetics in which the rotation of the aromatic ring is severely restricted (FIG. 6). The CO—Cα-Cβ-Cγ (arom) dihedral angle of the phosphotyrosine residue in the crystal structure of Stat3 (Becker et al, 1998) is 174 deg (FIG. 7), and those of the cinnamate and indole-2-carboxylate are approximately 180 deg. Thus the tyrosine replacements hold the aromatic ring in rigid conformations optimal for binding interactions.

The use of cinnamide as a tyrosine replacement in inhibitors of Src-family SH2 domains was reported by Shahripour et al. (1996). In this paper the activity of the lead was reduced 7-10-fold by replacing phosphotyrosine with 4-phosphoryloxycinnamide. McKinney et al, (2000, 2001) used this pTyr mimic in inhibitors of Stat4 and Stat6. Vu et al., (1999) reported the use of the 3-phosphoryloxyindole unit in development of inhibitors of the SH2 domain of Zap70.

Because phosphopeptides are weak drug candidates due to cleavage of the phosphate group by phosphatases, the phosphoryloxy group was replaced with the isosteric difluoromethylphosphono (F2Pm) group (Wrobel and Dietrich 1993) (compounds 8, 12, 16, 19). The difluoromethyl group renders this phosphate mimic stable to phosphatases. McKinney et al, (2000, 2001) also used 4-phosphonodifluoromethylcinnamide in inhibitors of Stat4 and Stat6. An aspect of the present disclosure is a synthetic route to 4-phosphonodifluoromethylcinnamate for incorporation into the peptidomimetics that is more efficient than those reported in McKinney et al. This scheme, called Scheme 1, is shown in FIG. 13. The affinity decreases by an order of magnitude when the difluoromethylphosphonate replaces the phosphate. Phosphate substitution by difluoromethylphosphonate has been shown to reduce affinity in inhibitors of other SH2 domains (Burke et al. 1994).

SAR studies indicated that cyclohexylalanine in place of leucine enhanced activity so this non-natural amino acid was incorporated into several mimetics (compounds 7-9, 15-17).

Examination of a model of Ac-pTyr-Leu-Pro-Gln-NH₂, SEQ ID NO:14 docked into the Stat3 SH2 domain suggested that the Leu-Pro dipeptide unit could be substituted with a fused ring or bicyclic lactam dipeptide mimic such as an amino-azabicyclononane carboxylate (reviewed by Gillespie et al., 1997). Replacing the central two amino acids with Haic (5-(amino)-1,2,4,5,6,7-hexahydro-4-oxo-(2S,5S)-azepino[3,2,1-hi]indole-2-carboxylic acid) results in inhibitors with high affinity (compounds 10-13). The IC₅₀ values for these compounds range from 100-200 nM. Haic, being a heterocycle, has much less peptidic character than peptide 1, and thus is expected to render the inhibitors more stable to proteolysis and completely stable to cis/trans proline isomerism. The Haic unit is a rigid scaffold that serves to present the aryl phosphonate and alkylcarboxamide functional groups in high affinity orientations for binding to Stat3. Haic has been employed in programs to develop proteolytic enzyme inhibitors as well as antagonists of angiotensin and bradykinin (Amblard et al., 1999), but to the knowledge of the present inventors, has not been used in SH2 domain or Stat inhibitor development programs.

It is a further interesting aspect of the disclosure that replacing proline with 3,4-methanoproline enhanced activity (compounds 14-17).

Substitution of glutamine generally produces peptides with reduced activity. However, compound 18, incorporating pyrrolidinoacetamide at pY+3, exhibited an IC₅₀ value around 800 nM. Compounds 19 and 20 represent the first totally non-peptide inhibitors Stat3.

The threonine and valine residues can be replaced by groups as small as methyl groups. The benzyl group was chosen to enhance cell penetration (compounds 7-9, 11-13, 15-17).

Design of Cell-Penetrable Inhibitors of Stat3.

A series of phosphopeptides with the mts sequences and a pro-drug of compound 12 was prepared and assayed for the ability to inhibit Stat3 activity in cell culture.

| | |
|---|---|
| Peptide | Sequence |

| | |
|---|---|
| 21 | F2PmCinn-Leu-Pro-Gln-Thr-Val-mts, |
| | SEQ ID NO:15 |
| 22 | Ac-F2Pmp-Leu-Pro-Gln-Thr-Val-mts, |
| | SEQ ID NO:16 |
| 23 | F2Pm(POM)Cinn-Haic-Gln-NHBn |
| 24 | Ac-pTyr-Leu-Pro-Gln-Thr-Val-mts, |
| | SEQ ID NO:17 |
| 25 | Ac-Tyr-Leu-Pro-Gln-Thr-Val-mts, |
| | SEQ ID NO:18 |
| 26 | H-Pro-Tyr-Leu-Lys-Thr-Lys-Phe-Ile-mts, |
| | SEQ ID NO:19 |
| 27 | H-Pro-pTyr-Leu-Lys-Thr-Lys-Phe-Ile-mts, |
| | SEQ ID NO:20 |
| 28 | F2PmCinn-Haic-Gln-Thr-mts, |
| | SEQ ID NO:21 |

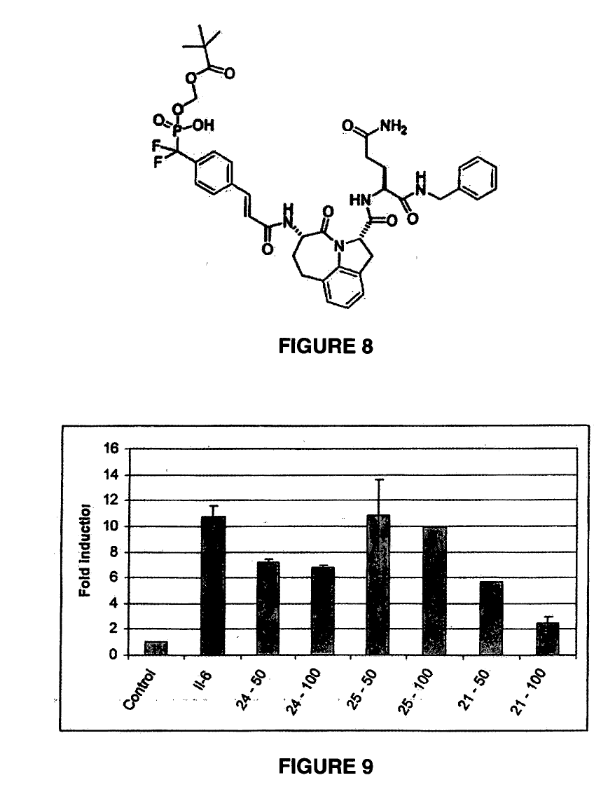

Peptide 24 is mts attached to peptide 1 and peptide 25 is the non-phosphorylated control. In peptide 21 the phosphotyrosine is replaced with the phosphonodifluoromethylcinnamide unit to impart stability to proteases and phosphatases. Peptide 22 is the lead peptide in which the phosphotyrosine was replaced with 4-phosphonodifluoromethylphenylalanine (F2Pmp), which is a phosphatase-stable pTyr mimic (Burke et al, 1994; Wrobet and Dietrich, 1993). Peptides 26 and 27 are those reported by Turkson et al (2001) to inhibit Stat3 activity in cell culture. The mimetic F2PmCinn-Haic-Gln-NHBn has a negatively charged phosphono group that is expected to impede passive diffusion across the non-polar cell membrane. For compound 23 a pivaloyloxymethyl (POM, Farquhar) group was added to one of the phosphonyl oxygen atoms of 12 to give F2PmCinn(POM)-Haic-Gln-NHBn (FIG. 8).

Biological Evaluation of Peptidomimetic Pro-Drug and cell Penetrable Peptides

The compounds were evaluated using HepG2 or HEP3B hepatoma cells. On stimulation with IL-6 there is a dramatic increase in phosphorylation of Stat3. The phosphoStat3 migrates to the nucleus and initiates transcription of acute phase response genes, such as α2-macroglobulin. HEPG2 and HEP3B cells are easily transfected with reporter gene plasmids and are easy to culture. Thus these cell lines are ideal test systems to evaluate Stat3 inhibitors.

The series of peptides and F2PmCinn(POM)-Haic-Gln-NHBn were evaluated for the ability to, (i) inhibit the IL-6 stimulated expression of a firefly luciferase reporter gene under control of the Stat3-responsive α2-macroglobulin promoter, and (ii) inhibit IL-6 stimulated Stat3 migration to the nucleus in HepG2 cells.

Inhibition of Luciferase Expression in Hepatoma Cells

Liver hepatoma cells, HEPG2, when stimulated with IL-6, respond by increased phosphorylation of Stat3, which translocates to the nucleus and initiates transcription of acute response phase genes, such as a2-macroglobulin. HEPG2 cells were transfected with luciferase gene construct containing the α2-macroglobulin promoter. Cells were treated with peptides 21, 24, and 25 for 1 hr before stimulation with IL-6. Four hours later cells were lysed and luciferase activity was assayed (FIG. 9). In the initial screen neither phosphopeptide 26 nor the unphosphorylated version, 27 (Turkson et al, 2001), showed inhibition of luciferase activity at 100 μM. Thus the disclosed peptides are more potent than those of Turkson et al. The control peptide, 25, showed no significant reduction in luciferase activity. Phosphopeptide 24 reduced induction to 60-70% of that of IL-6 at 50 and 100 μM. However, peptide 21, possessing the phosphonodifluoromethyl cinnamate mimic, reduced luciferase activity to 50 and 25% of untreated cells at concentrations of 50 and 100 μM, respectively.

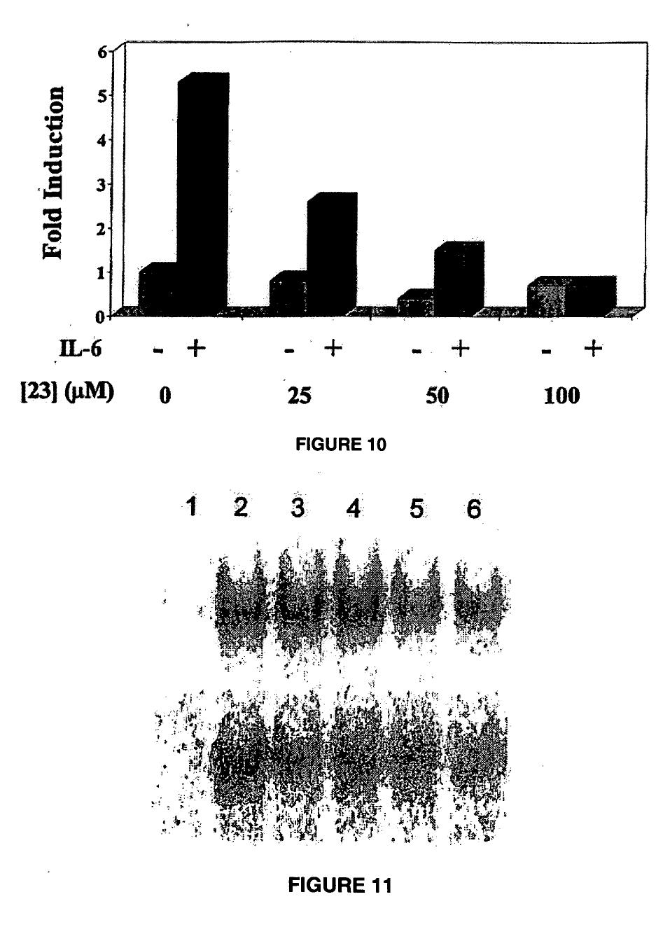

Peptidomimetic 23 was tested for the ability to inhibit luciferase induction in a second hepatoma cell line: Hep3B. Identical procedures as for the HEPG2 experiments above were used in this cell line. FIG. 10 shows a dose dependant reduction in luciferase activity.

Inhibition of Stat3 Nuclear Translocation

HepG2 cells were treated with 100 μM peptide for 1 hr. Cells were stimulated with IL-6 and 15 min later cells were lysed and nuclear extracts were obtained. Electrophoretic mobility shift assays (FIG. 11) showed that the stabilized F2PmCinn-mts peptide (21, lane 5) and the prodrug (23, lane 6) inhibited Stat3 translocation to the nucleus.

The luciferase inhibition experiments show that Peptides 21, 24, and the POM pro-drug 23 are inhibitors of Stat3 activation, translocation to the nucleus, and expression of Stat3 responsive genes. Inhibition of translocation to the nucleus is shown in the EMSA assay of FIG. 11. These biochemical experiments demonstrate that peptidomimetics based on peptide 1 are useful reagents for the study of Stat3 signaling in cancers of the breast, prostate, ovary, brain, pancreas, head and neck, melanoma, myeloma, lymphoma, etc., as well as development, immunology, and other fields. The Haic compounds are also useful as pharmaceutical agents or as the basis for the design of future agents. Compounds 11, 12, and 13 are unique in that they contain only one natural amino acid, glutamine.

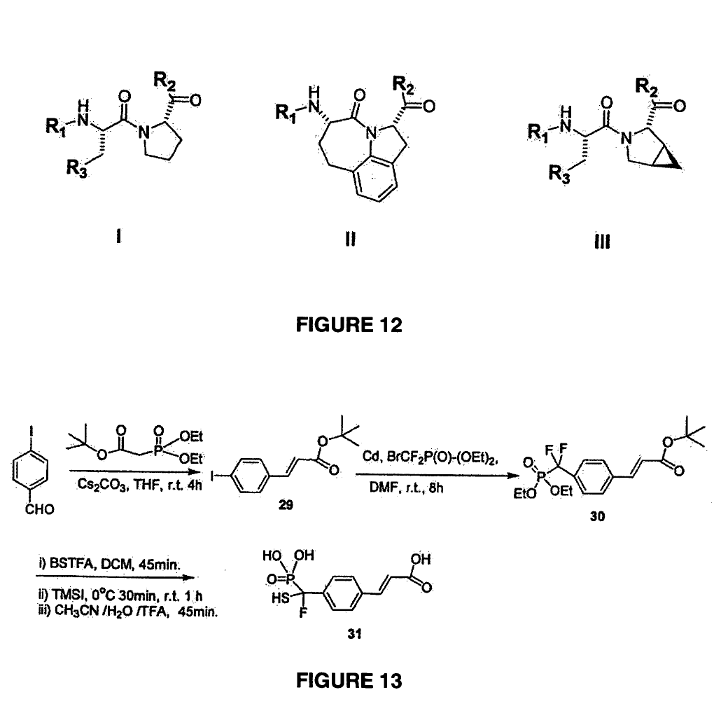

The disclosed peptidomimetics are based on three main scaffolds derived from the Leu Pro dipeptide central unit of peptide 1 (FIG. 12). Type I is Xxx-Pro, in which Xxx is leucine (R₃=isopropyl) or cyclohexyl alanine (R₃=cyclohexyl). Type II is Haic. Type III is Xxx-3,4-methanoPro, in which Xxx is leucine (R₃=isopropyl) or cyclohexyl alanine (R₃=cyclohexyl). In all cases, R¹ is a phosphotyrosine or phosphotyrosine mimic, and R₂ is glutamine, a glutamyl peptide, or a glutamine mimic.

Procedures

N^{α}-protected amino acids were purchased from Advanced Chemtech, NovaBiochem, ChemImpex, or AnaSpec. HOBt was from ChemImpex. Fmoc-Haic was obtained from ChemImpex or Neosystems. DMF for amino acid solutions was Baker dried. Other solvents were reagent grade and were used without further purification. Peptides were purified by reverse phase HPLC on a Rainin Rabbit HPLC using a Vydac 2.5×25 cm C18 column. Gradients of ACN in H₂O (both containing 0.1% TFA) or MeOH in 0.01 M NH₄OAc (pH 6.5) at 10 mL/min were employed. Peptides were tested for purity by reverse phase HPLC on Hewlett Packard 1090 HPLC or an Agilent 1100 HPLC using a Vydac 4.6×250 mm C18 peptide/protein column in two systems: A. 10-80% CAN/30 min in which both H₂O and ACN contained 0.1% TFA; B. 10-80% MeOH in 0.01 M NH₄OAc. Both gradients were run at 1.5 mL/min and detection at 230 nm and 275 nm was performed simultaneously.

Representative solid phase and solution phase syntheses are given below. Characterization by MS and in most cases NMR of all compounds was performed.

Preparation of Rink-polyamide solid phase peptide synthesis support.

PL-DMA resin (Polymer Laboratories, Ltd) was derivatized with ethylenediamine as described (Arshady et al., 1981). The resin was then functionalized by the addition of 3 eq. of p-[(R,S)-α-[1-(9H-fluoren-9-yl)-methoxyformamido]-2,4-dimethoxybenzyl]-phenoxyacetic acid (Rink linker) in the presence of 3 eq. each of diisopropylcarbodiimide (DIPCDI) and 1-hydroxybenzotriazole (HOBt).

SEQ ID NO:22

Synthesis of Ac-pTyr-Leu-Pro-G1-Thr-mts, 25

Rink-derivatized PL-DMA resin (0.9454 g, ca 0.09 mmol) was used to assemble the peptide up to the leucine at pY+1. Fmoc-amino acids, HOBt, and diisopropylcarbodiimide (DIPCDI) were added in 10-fold excess in 1:1 DMF/CH₂Cl₂ and couplings were monitored with ninhydrin. Couplings were complete in 1 hr. The Fmoc group was removed with a solution of 20% piperidine and 2% diazabicycloundecane (DBU) in DMF for 5 min and 26 min treatments. Side chain protection of threonine was tert-butyl and for glutamine, trityl. When assembly was complete the resin was split into four equal aliquots. One of these was acylated with 10-fold excess of Fmoc-Tyr(PO₃tBu₂)-OH and DIPCDI/HOBt as before. Fmoc-Tyr(PO₃tBu₂)-OH was synthesized just prior to use by adding two eq. of N,N-diisopropyl di-tert-butylphosphoramidite and tetrazole to Fmoc-Tyr-OH for two hr followed by 30 min oxidation with 10 eq of tert-butylhydroperoxide. After aqueous Na₂S₂O₆ washing the solvent was evaporated, and the residue washed with hexane. The residue was dissolved in DMF and added to the resin. After 1 hr ninhydrin indicated complete reaction. The Fmoc group was removed and the amino terminus was capped with acetic anhydride. After assembly of the peptide the resin was treated with 3×10 ml of trifluoroacetic acid/water/triisopropylsilane (TFA/H₂O/TIS 95:2.5:2.5) for 10 min each. The combined filtrates sat for 2 hr and the volume was taken down in vacuo. The solution was dropped into ice cold Et₂O and the precipitate was collected by filtration and washed 2× more with Et₂O to give 114 mg of white solid. The peptide was purified by reverse phase HPLC to give 36 mg of peptide. HPLC System A 20.27 mm ESI MS (M+2H) Calc'd 986.64 Found 986.1

SEQ ID NO:23

Synthesis of Ac-Tyr-Leu-Pro-Gln-Thr-mts, 24

The same procedure was used as in synthesis of peptide 25, except that Fmoc-Tyr(tBu)-OH was used to incorporate tyrosine. Crude yield, 99 mg. Yield after purification, 50 mg. HPLC System A 21.99 mm ESI MS (M+2H) Calc'd 946.65 Found 946.1

4-(phosphonondifluoromethyl)-cinnamic acid was synthesized as shown in FIG. 13. A solution of tert-butyl diethylphosphonoacetate (1.0 g, 3.96 mmol), 4-iodobenzaldehyde (0.920 g, 3.96mmol) and cesium carbonate (1.93 g, 5.94 mmol) in dry THF (15 mL) was stirred for 4 h. The solvent was removed in vacuo and the residue dissolved in 100 mL of EtOAc. This solution was washed with water (2×20 mL) and brine (1×20 mL) and dried over MgSO₄. After filtration and concentration the crude product was purified by silica gel chromatography eluting 10% EtOAc-Hexane. Desired white solid 29 was obtained with 86% yield (1.11 g). ¹H NMR (CDCI₃, 300 MHz) δ 7.70 (d, 1H, J=8.4 Hz), 7.48 (d, 1H, J=15.9 Hz), 7.21 (d, 2H, J=8.4 Hz), 6.36 (d, 1H, J=16.2 Hz), 1.53 (s, 9H).

To a solution of diethyl bromodifluoromethylphosphonate (0.645 g, 2.41 mmol) in dry DMF (10 mL),cadmium powder (0.541 g, 4.82 mmol) was added. The suspension was stirred for 3 h under argon atmosphere. The unreacted cadmium was removed by filtration under argon and the filtrate was treated with CuCl (0.286 g, 2.89 mmol) and 29 (0.500 g, 1.51 mmol) at room temperature for 8h. The mixture was diluted with 100 mL of Et2O, stirred for 5 min and filtered. The organic solution was washed with saturated NH₄Cl (2×20 mL) and water (3×20 mL), dried over MgSO₄ and evaporated to give an oily residue. The residue was purified by silica gel column chromatography with 40% EtOAc-hexane to give 0.492 g (83%) of 30 as a colorless oil.

¹H NMR (CDCl₃, 300 MHz) δ 7.56-7.64 (m, 5H), 6.43 (d, 1H, J=16.02 Hz), 4.15-4.25 (m, 4H), 1.54 (s, 9H), 1.32 (t, 6H, J=7.05 Hz).

To a solution of 30 (0.400 g, 1.02 mmol) in 10 mL dry CH₂Cl₂ was added bis(trimethylsilyl)trifluoroacetamide (0.300 mL, 1.12 mmol). After 45 min., the mixture was cooled to 0° C. and iodotrimethylsilane (0.700 mL 5.1 mmol) was added dropwise. Stirring was continued for 30 min. at 0° C. and 1 h at room temperature. The solution was concentrated in vacuo. The residue was dissolved in 10 mL ACN/H₂O/TFA (10:5:4), stirred for additional 45 min., and the solvents evaporated in vacuo. Toluene was added and evaporated twice. After adding Et₂O, the solids were collected by filtration and washed successively with Et₂O and CH₂Cl₂ to give 31 as a white powder. Yield: 0.250 g (89%). ¹H NMR (DMSO-d₆, 300 MHz) δ 7.71 (d, 2H, J=8.14 Hz), 7.54 (d, 1H, J=16.05 Hz), 7.45 (d, 2H, J=8.09 Hz), 6.53 (d, 1H, J=16.04 Hz).

SEQ ID NO:24

Synthesis of 4-(phosphonondifluoromethyl)-cinna-
moyl-Leu-Pro-Gln-Thr-mts, 21

A reactor vessel on an Advanced Chemtech 348 automated peptide synthesizer was charged with 0.2 gm of Rink-linker-derivatized PL-DMA resin (ca 0.18 mmole). The sequence Ala-Ala-Val-Leu-Leu-Pro-Val-Leu-Leu-Ala-Ala-Pro-NH₂, SEQ ID NO:11 was assembled using automated coupling and deprotection protocols. N^{α}-Fmoc protected amino acids were dissolved to a concentration of 0.5M in DMF containing 0.5 M 1-hydroxybenzotriazole (HOBt). Coupling was achieved using a 110-fold molar excess of Fmoc-amino acid by adding equal volumes of amino acid/HOBt and 0.5 M diisopropylcarbodiimide in CH₂Cl₂ to the resin and agitating for one hour. The resin was drained and washed 5× with DMF/CH₂Cl₂ (1:11). Fmoc group removal was achieved by treating the resin with 7 ml of a solution of 2% DBU and 20% piperidine in DMF for 5 minutes, draining the resin, and treating again with 7 ml of the same solution for 25 min. The resin was drained and washed 5× with DMF/CH₂Cl₂ (1:1). Leu-Pro-Gln(Trt)-Thr(tBu)-Val was added by manual coupling of 10 Eq. of Fmoc-amino acid, DIPCDI, and HOBt and deprotection with the same protocol. On completion of the sequence, 3 equivalents each of 4-(phosphonondifluoromethyl)-cinnamic acid, PyBOP, and HOBt plus 6 equivalents of diisopropylethylamine in DMF/CH₂Cl₂ (1:1) were added to the resin. After overnight agitation the resin was drained, washed with DMF/CH₂Cl₂ (1:1) and CH₂Cl₂. The resin was treated with 3×10 ml of TFA/H₂O/TIS (95:2.5:2.5) for 10 min each. The combined filtrates sat for 2 hr more and the volume was taken down in vacuo. The solution was dropped into ice cold Et₂O and the precipitate was collected by filtration. The peptide was purified by reverse phase HPLC to give mg of 21. ESI-MS (M+2H) calc'd 973.12 Found 973.3 (M+2H)

Synthesis of Ac-pTyr-Haic-Gln-Thr-NH₂, 10

Rink resin (0.2 gm, 0.6 mmol/gm, 0.12 mmol) was washed with DMF/DCM (1:1). It was treated with 5 ml of 20% piperidine in DMF for 5 minutes, drained, and re-treated with 5 ml of the piperidine solution. The resin was drained and washed 5× with 5 ml of DMF/DCM. Fmoc-Thr(^{t}Bu)-OH (0.165 gm, 0.36 mmol), HOBt (0.055 gm, 0.36 mml), and diisopropylcarbodiimide (.053 ml, 0.36 mmole) in ca 5 ml of DMF/DCM were added and the resin was agitated by bubbling N₂. When the ninhydrin test of the resin was negative the resin was drained and washed with 5×5 ml of DMF/DCM. The Fmoc group was removed with 2×5 ml of 20% piperidine in DMF for 3 and 7 min each and the resin was washed with 5x 5 ml of DMF/DCM. Fmoc-Gln(Trt)-OH and Fmoc-Haic-OH were coupled to the growing peptide chain in the same manner as the first amino acid. Fmoc-Tyr(PO₃H₂)—OH (0.184 gm, 0.36 mmole), PyBOP (0.184 gm, 0.36 mmol), HOBt (0.055 gm, 0.36 mml) and diisopropylethylamine (0.172 ml, 0.72 mol) in 5 ml of DMF/DCM were added to the resin and the resin was again agitated with N₂. After a negative ninhydrin test of the resin it was drained and the Fmoc group removed as before. The resin was capped with acetic anhydride and Et₃N until a negative ninhydrin test was obtained. The resin was washed with DMF/DCM followed by DCM and dried in vacuo. The resin was treated with 3×10 ml of TFA:H₂O:TIS (95:2.5:2.5) (TIS=triisopropylsilane) for 10 mm each. The combined filtrates were evaporated after 1 ½ hr. The residue was dropped into ice cold Et₂O and the solid collected by centrifugation. After washing with Et₂O 2× more the solid was dried to give 66 mg of crude peptide. The product was purified by reverse phase HPLC using a gradient of ACN in H₂O in which both solvents contained 0.1% TFA to give 12.5 mg of 10. The peptide was >98% pure as judged by analytical HPLC in ACN/H₂O (0.1% TFA) and MeOH/0.0 1 M NH₄OAc. ESI-MS (M+H) calc'd 760.7 Found 760.3

Synthesis of F2PmCinn-Haic-Gln-NHBn, 12

Rink Resin (0.2 gm ca 0.18 mmol) was swollen in DMFCH₂Cl₂ (1:1) and was treated with 5 ml 20% piperidine in DMF for 3 min and again for 7 min. The resin was washed with DMFCH₂Cl₂ (1:1) 7× and 3 eq. each of Fmoc-Gln-NHBn, HOBt, and DIPCDI were added in 5 mL of DMFCH₂Cl₂ (1:1). When the resin tested negative in the ninhydrin test, it was drained, washed 5× with DMFCH₂Cl₂ (1:1) and then deprotected with 20% piperidine as before. Fmoc Haic-OH was coupled and deprotected as before. The resin was then treated with 3eq. of 4-phosphonodifluoromethylcinnamic acid, PyBOP, HOBt and 6 eq of DIPEA. When ninhydrin was negative the resin was wached with DMFCH₂Cl₂ (1:1), DCM and dried. The resin was treated with 3×10 ml of TFA:H₂0:TIS for 10 min each. The combined filtrates were evaporated after 1 ½ hr. The residue was dropped into ice cold Et₂O and the solid collected by centrifugation. After washing with Et₂O 2× more the solid was dried and purified by reverse phase HPLC using a gradient of ACN in H₂O in which both solvents contained 0.1% TFA. The peptide was >98% pure as judged by analytical HPLC in ACN/H₂O (0.1% TFA) and MeOH/0.01 M NH₄OAc. Yield ESI-MS (M+H) Calc, 724.66; Found: 724.5. NMR see accompanying spread sheet.

Preparation of F2PmCinn(POM)-Haic-Gln-NHBn. 23

Resin (0.2 g) containing F2PmCinn-Haic-Gln-NHBn was treated with 10 eq of iodomethyl pivalate and 10 eq of DIPEA. at 80° C. for 4 hr. The peptide was cleaved with TFA:TIS:H₂O as above and the product isolated by evaporation of the solvents and washing with Et₂O. The compounds were purified with reverse phase HPLC to give 31 mg of 23. ESI-MS (M+H) Calc'd: 838.8. Found: 838.6

Fluorescence Polarization Assays

Aliquots of 50 μl of 80 nM full length Stat³a and 20 nM of probe in 50 mM NaCl, 10 mM Hepes, 1 mM Na4EDTA, 2 mM DTT, and 1% NP-40 were placed in wells of a 96 well black, opaque, non-stick microtiter plate. To each well was added 50 μl of peptide solutions of decreasing concentration in the same buffer. In some cases dilutions were prepared manually and in some dilutions were prepared on a Perkin Elmer 204DT liquid handling robot. Fluorescence polarization was then read in a Tecan Polarian plate reader. Each concentration was run in duplicate and the average value mP versus inhibitor concentration were plotted and IC₅₀ values were obtained.

Inhibition of Luciferase Reporter Gene

Cell culture and transient transfection. HepG2 cells were grown in DMEM containing 10% fetal bovine serum. Cells were plated at a density of 3×10⁵ per 6cm dish. Next day, plasmids were transfected with a plasmid comprised of firefly luciferase under the α2-macroglobulin promoter (Ren & Schaefer, 2002) at a ratio of 1 μg DNA to 3 μl Fugene-6 reagent according to the protocol supplied by Roche. After 48 hours, cells were treated with different peptides in serum free media for 1 hour, and then stimulated with 6 ug/ml IL-6. Cells were harvested at the time indicated.

Luciferase assay. Luciferase assay reagents were purchased from Promega and the assays performed according to the manufacturer's protocol. In brief, cells were washed twice with ice-cold PBS and then collected. After centrifugation at 4,500 rpm for 1 minute, cells were lysed in 1× lysis buffer (Promega) at room temperature for 30 minutes. The lysate was cleared by centrifugation at 13,000 rpm for 10 minutes. Then 50 μl of luciferase substrate was added to 10 μl of supernatant and the luciferase value was read by luminometer (Pharmingen). Each transfection was normalized to concomitant β-galactosidase expression from a control transfected plasmid pYN3214-lacZ.

Electrophoretic Mobility Shift Assays

Nuclear extract was extracted as previously described (Andrews and Faller, 1991). Equal amount of nuclear extract protein was then incubated with ³²P labeled high-affinity c-sis-inducible element (hSIE; 5′-GTGCATTTCCCGTAAATCTTGTCTACA-3′ , SEQ ID NO:25) (Santa Cruz). The reactions were performed in a total volume of 24 μl in buffer consisting of 10 mM HEPES (pH 7.8), 50 mM KCl, 1 mM EDTA, 5 mM MgCl₂, 10% glycerol, 5 mM dithiothreitol, 1 mg of bovine serum albumin per ml, 0.5 mM phenyl-methylsulfonylflouride, and 1 mM Na₃VO₄ with 1 μg of poly (dI-dC) and 0.3 ng of ³²P-labeled hSIE. Following incubation for 15 min at room temperature, the reactions were electrophoresed on 4% native polyacrylamide gets. The gels were dried and exposed to phosphoimager screen (Molecular Dynamics). The screen was then scanned and the bands showing Stat3-DNA binding were quantified using the Storm System (Molecular Dynamics).

Pharmaceutically Acceptable Carriers

Aqueous compositions of the present disclosure comprise an effective amount of peptide or peptide mimetic dissolved and/or dispersed in a pharmaceutically acceptable carrier and/or aqueous medium.

The phrases “pharmaceutically and/or pharmacologically acceptable” refer to molecular entities and/or compositions that do not produce an adverse, allergic and/or other untoward reaction when administered to an animal, and/or a human, as appropriate.

As used herein, “pharmaceutically acceptable carrier” includes any and/or all solvents, dispersion media, coatings, antibacterial and/or antifungal agents, isotonic and/or absorption delaying agents and/or the like. The use of such media and/or agents for pharmaceutically active substances is well known in the art. Except insofar as any conventional media and/or agent is incompatible with the active ingredient, its use in the therapeutic compositions is contemplated. Supplementary active ingredients can also be incorporated into the compositions.

The active compounds may generally be formulated for parenteral administration, e.g., formulated for injection via the intravenous, intramuscular, sub-cutaneous, intralesional, and/or even intraperitoneal routes. The preparation of an aqueous composition that contains an effective amount of peptide or peptide mimetic agent as an active component and/or ingredient will be known to those of skill in the art in light of the present disclosure. Typically, such compositions can be prepared as injectables, either as liquid solutions and/or suspensions; solid forms suitable for using to prepare solutions and/or suspensions upon the addition of a liquid prior to injection can also be prepared; and/or the preparations can also be emulsified.

The pharmaceutical forms suitable for injectable use include sterile aqueous solutions and/or dispersions; formulations including sesame oil, peanut oil and/or aqueous propylene glycol; and/or sterile powders for the extemporaneous preparation of sterile injectable solutions and/or dispersions. In all cases the form must be sterile and/or must be fluid. It must be stable under the conditions of manufacture and/or storage and/or must be preserved against the contaminating action of microorganisms, such as bacteria and/or fungi.

Solutions of the active compounds as free base and/or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils. Under ordinary conditions of storage and/or use, these preparations contain a preservative to prevent the growth of microorganisms.

The disclosed peptides and peptide mimetics or analogs of the present disclosure can be formulated into a composition in a neutral and/or salt form. Pharmaceutically acceptable salts, include the acid addition salts (formed with the free amino groups of the peptide) and/or which are formed with inorganic acids such as, for example, hydrochloric and/or phosphoric acids, and/or such organic acids as acetic, oxalic, tartaric, mandelic, and/or the like. Salts formed with the free carboxyl groups can also be derived from inorganic bases such as, for example, sodium, potassium, ammonium, calcium, and/or ferric hydroxides, and/or such organic bases as isopropylamine, trimethylamine, histidine, procaine and/or the like. In terms of using peptide therapeutics as active ingredients, the technology of U.S. Pat. Nos. 4,608,251; 4,601,903; 4,599,231; 4,599,230; 4,596,792; and/or 4,578,770, each incorporated herein by reference, may be used.

The carrier can also be a solvent and/or dispersion medium containing, for example, water, ethanol, polyol (for example, glycerol, propylene glycol, and/or liquid polyethylene glycol, and/or the like), suitable mixtures thereof, and/or vegetable oils. The proper fluidity can be maintained, for example, by the use of a coating, such as lecithin, by the maintenance of the required particle size in the case of dispersion and/or by the use of surfactants. The prevention of the action of microorganisms can be brought about by various antibacterial and/or antifungal agents, for example, parabens, chlorobutanol, phenol, sorbic acid, thimerosal, and/or the like. In many cases, it will be preferable to include isotonic agents, for example, sugars and/or sodium chloride. Prolonged absorption of the injectable compositions can be brought about by the use in the compositions of agents delaying absorption, for example, aluminum monostearate and/or gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the required other ingredients from those enumerated above. In the case of sterile powders for the preparation of sterile injectable solutions, the preferred methods of preparation are vacuum-drying and/or freeze-drying techniques which yield a powder of the active ingredient plus any additional desired ingredient from a previously sterile-filtered solution thereof. The preparation of more, and/or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like can also be employed.

For parenteral administration in an aqueous solution, for example, the solution should be suitably buffered if necessary and/or the liquid diluent first rendered isotonic with sufficient saline and/or glucose. These particular aqueous solutions are especially suitable for intravenous, intramuscular, subcutaneous and/or intraperitoneal administration. In this connection, sterile aqueous media which can be employed will be known to those of skill in the art in light of the present disclosure. For example, one dosage could be dissolved in 1 ml of isotonic NaCl solution and/or either added to 1000 ml of hypodermoclysis fluid and/or injected at the proposed site of infusion, (see for example, “Remington's Pharmaceutical Sciences” 15th Edition, pages 1035-1038 and/or 1570-1580). Some variation in dosage will necessarily occur depending on the condition of the subject being treated. The person responsible for administration will, in any event, determine the appropriate dose for the individual subject.

The peptides and/or agents may be formulated within a therapeutic mixture to comprise about 0.0001 to 1.0 milligrams, and/or about 0.001 to 0.1 milligrams, and/or about 0.1 to 1.0 and/or even about 10 milligrams per dose and/or so. Multiple doses can also be administered.

In addition to the compounds formulated for parenteral administration, such as intravenous and/or intramuscular injection, other pharmaceutically acceptable forms include, e.g., tablets and/or other solids for oral administration; liposomal formulations; time release capsules; and/or any other form currently used, including cremes.

One may also use nasal solutions and/or sprays, aerosols and/or inhalants in the present invention. Nasal solutions are usually aqueous solutions designed to be administered to the nasal passages in drops and/or sprays. Nasal solutions are prepared so that they are similar in many respects to nasal secretions, so that normal ciliary action is maintained. Thus, the aqueous nasal solutions usually are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation. Various commercial nasal preparations are known and/or include, for example, antibiotics and/or antihistamines and/or are used for asthma prophylaxis.

Additional formulations which are suitable for other modes of administration include vaginal suppositories and/or pessaries. A rectal pessary and/or suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent methyl and/or propylparabens as preservatives, a dye and/or flavoring, such as cherry and/or orange flavor.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and structurally related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Stark, G. R.; Kerr, I. M.; Williams, B R.; Silverman, R. H; Schreiber, R. D. How cells respond to interferons Ann. Rev. Biochem. 1998, 67, 227-264.
Bromberg, J. and Darnell, J. E., Jr. The rote of STATs in transcriptional control and their impact on cellular function Oncogene 2000, 19, 2468-2473.
Levy, D. E.; Darnell, J. E., Jr Stats: transcriptional control and biological impact Nature Rev. Mo!. Cell. Blot. 2002, 3, 651-662.
Akira, S.; Nishio, Y.; lnoue, M.; Wang, X. J.; Wei, S.; Matsusaka, T.; Yoshida, K.; Sudo, T.; Naruto, M.; Kishinioto, T. Molecular cloning of APRF, a novel IFN-stimutated gene factor 3 p91-related transcription factor involved in the gp130-mediated signaling pathway Cell 1994, 77, 63-71
Zhong; Z.; Wen, Z.; Darnell Jr., J. E. Stat3: A STAT family member activated by tyrosine phosphorylation in response to epidermat growth factor and interleukin-6 Science, 1994a, 264, 95-98
Zhong; Z.; Wen, Z.; Darnell Jr., J. E. Jr Stat3 and Stat4: Members of the Family of Signal Transducers and Activators of Transcription Proc. Nat. Acad. Sci., U.S.A. 1994b, 91, 4806-4810
Stahl, N.; Farruggella, T. J.; Boulton, T. G.; Zhong, Z.; Darnell, J. E., Yancopoulos, G. D. Choice of STATs and other substrates specified by modular tyrosine-based motifs in cytokine receptors Science 1995, 267, 1349-1353
Gerhartz, C.; Heesel, B.; Sasse, J.; Hemmann, U.; Landgraf, C.; Schneider-Mergener, J.; Horn, F.; Heinrich, P. C.; Graeve, L. Differential activation of acute phase response factor/STAT3 and STAT1 via the cytoplasmic domain of the interleukin 6 signal transducer gp130. I. Definition of a novel phosphotyrosine motif mediating STAT1 activation. J. Biol. Chem. 1996, 271, 12991-12998
Becker, S.; Gromer, B.; Muller, C. W. Three-dimensional structure of the Stat3β homodimer bound to DNA Nature, 1998, 394, 145-151.
Coffer, P.; Kruijer, W. EGF receptor deletions define a region specifically mediating STAT transcription factor activation. Biochem. Biophys. Res. Commun. 1995, 210, 74-81
Zhang, T.; Ma, J.; Cao, X. Grb2 regulates Stat3 activation negatively in epidermal growth factor signaling. Biochemical J. 2003, 376, 457-464.
Schreiner, St. J.; Schiavone, A. P.; Smithgall, T. E. Activation of STAT3 by the Src family kinase Hck requires a functional SH3 domain J. Biol. Chem. 2002, 277, 45680-45687
Song, J. I.; Grandis, J. R. STAT signaling in head and neck cancer Oncogene 2000, 19, 2489-2495
Garcia, R.; Yu, C. L.; Hudnall, A.; Catlett, R.; Nelson, K. L.; Smithgall, T.; Fujita, D. J.; Ethier, S. P.; Jove, R. Constitutive activation of Stat3 in fibroblasts transformed by diverse oncoproteins and in breast carcinoma cells. Cell Growth Duff. 1997, 812, 1267-76
Schaefer, L. K.; Ren, Z.; Fuller, G. N.; Schaefer, T. S. Constitutive activation of Stat3.altpha. in brain tumors: Localization to tumor endothelial cells and activation by the endothelial tyrosine kinase receptor (VEGFR-2). Oncogene 2002, 21, 2058-2065.
Dhir, R.; Ni, Z.; Lou, W.; DeMiguel, F.; Grandis, J. R.; Gao, A C. Stat3 activation in prostatic carcinomas Prostate 2002, 51, 241-246.
Seki Y.; Suzuki N.; Imaizumi M.; Iwamoto T.; Usami N.; Ueda Y.; Hamaguchi M. STAT3 and MAPK in human lung cancer tissues and suppression of oncogenic growth by JAB and dominant negative STAT3. Int. J Oncology 2004, 24, 931-934.
Huang, M.; Page, C.; Reynolds, R. K.; Lin, J. Constitutive Activation of Stat 3 Oncogene Product in Human Ovarian Carcinoma Cells. Gynecol. Oncol. 2000, 79, 67-73.
Scholz, A.; Heinze, S.; Detjen, K. M.; Peters, M.; Welzel, M.; Hauff, P.; Schirner, M.;

Wiedenmann, B.; Rosewicz, S. Activated signal transducer and activator of transcription 3 (STAT3) supports the malignant phenotype of human pancreatic cancer. Gastroenterology 2003, 125, 891-905.
Benekti, M.; Baer, M. R.; Baumann, H.; Wetzler, M. Signal transducer and activator of transcription proteins in leukemias Blood 2003, 101, 2940-2954.
Weber-Nordt, R. M.; Egen, C.; Wehinger, J.; Ludwig, W.; Gouilleux-Gruart, V.; Mertelsmann, R.; Finke, J. Constitutive activation of STAT proteins in primary lymphoid and myeloid leukemia cells and in Epstein-Barr virus (EBV)-related lymphoma cell lines Blood 1996a, 88, 809-816
Bowman, T.; Garcia, R.; Turkson, J.; Jove, R. STATs in oncogenesis Oncogene 2000, 19, 2474-2488
Darnell, J. E., Jr. Transcription factors as targets for cancer therapy Nature Rev. Cancer 2002, 2, 740-749
Buettner, RaIf; Mora, Linda B.; Jove, Richard Activated STAT signaling in human tumors provides novel molecular targets for therapeutic intervention Clin. Cancer Res. 2002, 8, 945-954.
Bromberg, J. Stat proteins and oncogenesis. J. Clin. Invest. 2002, 109, 1139-1142.
Yu, H.; Jove, R. The status of cancer—new molecular targets come of age. Nature Rev. Cancer. 2004, 4, 97-105
Niu, G.; Wright, K. L.; Huang, M.; Song, L.; Haura, E.; Turkson, J.; Zhang, S.; Wang, T.; Sinibaldi, D.; Coppola, D.; Helter, R.; Ellis, L. M.; Karras, J.; Bromberg, J.; Pardoll, D.; Jove, R.; Yu, H. Constitutive Stat3 activity up-regulates VEGF expression and tumor angiogenesis. Oncogene 2002a, 21, 2000-2008.
Burke, W. M.; Jin, X.; Lin, H-J.; Huang, M.; Liu, R.; Reynolds, R. K.; Lin, J. Inhibition of constitutively active Stat3 suppresses growth of human ovarian and breast cancer cells Oncogene 2001, 20, 7925-7934
Catlett-Falcone, R., Landowski, T. H., Oshiro, M. M., Turkson, J., Levitzki, A., Savino, R., Ciliberto, G., Moscinski, L., Fernandez-Luna, J. L., Nunez, G., Dalton, W. S., and Jove, R. Constitutive activation of Stat3 signaling confers resistance to apoptosis in human U266 myeloma cells. Immunity 1999, 10/, 105-115.
Niu, G., Heller, R., Catlett-Falcone, R., Coppola, D., Jaroszeski, M., Dalton, W., Jove, R., and Yu, H. Gene therapy with dominant-negative Stat3 suppresses growth of the murine melanoma B16 tumor in vivo, Cancer Res. 1999, 59, 5059-5063.
Grandis, J. R., Drenning, S. D., Zeng, Q., Watkins, S. C., Melhem, M. F., Endo, S., Johnson, D. E., Huang, L., He, Y., and Kim, J. D. Constitutive activation of Stat3 signaling abrogates apoptosis in squamous cell carcinogenesis in vivo Proc Natl Acad Sci USA. 2000, 97, 4227-4232.
Grandis, Jennifer Rubin; Drenning, Stephanie D.; Chakraborty, Arup; Zhou, Min-Yu; Zeng, Qing; Pitt, Altysbn S.; Tweardy, David J. Requirement of Stat3 but not Stat1 activation for epidermal growth factor receptor-mediated cell growth in vitro. J. Clin. Invest. 1998, 102, 1385-1392
Niu, G.; Bowman, T.; Huang, M.; Shivers, S.; Reintgen, D.; Daud, A.; Chang, A.; Kraker, A.; Jove, R.; Yu, H. Roles of activated Src and Stat3 signaling in melanoma tumor cell growth Oncogene 2002b, 21, 7001-7010
Leong, P. L.; Andrews, G. A.; Johnson, D. E.; Dyer, K. F.; Xi, S.; Mai, J. C.; Robbins, P. D.; Gadiparthi, S.; Burke, N. A.; Watkins, S. F.; Grandis, J. R. Targeted inhibition of stat3 with a decoy oligonucleotide abrogates head and neck cancer cell growth Proc. Natl Acad. Sci. USA 2003, 100, 4138-4143Bowman, T.; Garcia, R.; Turkson, J.; Jove, R. STATs in oncogenesis Oncogene 2000, 19, 2474-2488
Real, P. J.; Sierra, A; de Juan, A.; Segovia, J. C.; Lopez-Vega, J. M.; Fernandez-Luna, J. L. Resistance to chemotherapy via Stat3-dependent overexpression of Bc1-2 in metastatic breast cancer cells Oncogene. 2002, 21, 7611-761 8.
Garcia, R.; Bowman, T. L.; Niu, G.; Yu, H.; Minton, S.; Muro-Cacho, C. A.; Cox, C. E.; Falcone, R.; Fairclough, R.; Parsons, S.; Laudano, A.; Gazit, A.; Levitzki, A.; Kraker, A.; Jove, R. Constitutive activation of Stat3 by the Src and JAK tyrosine kinases participates in growth regulation of human breast carcinoma cells Oncogene 2001, 20, 2499-2513.
Ren, Z., Cabell, L. A., Schaefer, T. S. and McMurray, J. S. Identification of a High Affinity Phosphopeptide Inhibitor of Stat3. Bioorg. Med. Chem. Lett. 2003, 13, 633-636.
Wrobel, J. and Dietrich, A. Preparation of L-(phosphodifluoromethyl) phenylalanine derivatives as non-hydrolyzable mimetics of O -phosphotyrosine, Tetrahedron Lett. 1993, 34, 3543-3546.
Rojas, M.; Donahue, J. P.; Tan, Z. J.; Lin, Y. Z. Genetic engineering of proteins with cell membrane permeability Nature Biotechnol. 1998, 16, 370-375.
Turkson J.; Ryan D.; Kim, J. S.; Zhang, Y.; Chen, Z.; Haura, E.; Laudano, A.; Sebti, S.; Hamilton, A. D.; Jove, R. Phosphotyrosyl peptides block Stat3-mediated DNA-binding activity, gene regulation and cell transformation J. Biol. Chem. 2001, 276, 45443-45455.
Shao, H.; Cheng, H. Y.; Cook, R. G.; Tweardy, D. J. Identification and Characterization of Signal Transducer and Activator of Transcription 3 Recruitment Sites within the Epidermal Growth Factor Receptor Cancer Res. 2003, 63, 3923-3930
Farquhar, David; Khan, Saeed; Srivastva, Devendra N.; Saunders, Priscilla P. Synthesis and Antitumor Evaluation of Bis[(pivaloyloxy)methyl] 2′-Deoxy-5-fluorouridine 5′-Monophosphate (FdUMP): A Strategy To Introduce Nucleotides into Cells J. Med Chem. 1994, 37, 3902-9
Blaskovich, M A.; Sun, J.; Cantor, A.; Turkson, J.; Jove, R.; Sebti, S.M. Discovery of JSI-124 (Cucurbitacin I), a selective janus kinase/signal transducer and activator of transcription 3 signaling pathway inhibitor with potent antitumor activity against human and murine cancer cells in mice Cancer Res 2003, 63, 1270-1279.
Sebti, S. M.; Jove, R. Cucurbitacin compounds, methods for treatment of cancer, and methods for identification of anticancer compounds. PCT Int. AppI. (2002), 53 pp. CODEN: PIXXD2 WO 2002078617 A2 20021010
Yu, H.; Pardoll, D.; Jove, R.; Dalton, W. Stat3 agonists and antagonists for therapy of inflammation, infection, immune disease and ischemia. PCT Int. Appt. (2002), 94 pp. CODEN: PIXXD2 WO 2002020032 Al 20020314
Karras, James G. Antisense oligonucleotide modulation of STAT3 expression. PCT Int. AppI. (2000), 104 pp. CODEN: PIXXD2 WO 2000061602
Karras, J. G.; McKay, R. A.; Lu, T.; Pych, J.; Frank, D. A.; Rothstein, T. L.; Monia, B. P. STAT3 regulates the growth and immunoglobulin production of BCL1 B cell lymphoma through control of cell cycle progression. Cellular Immunology 2000, 202, 124-135.
Jove, R.; Dalton, W.; Sebti, S.; Yu, H.; Helter, R.; Jaroszeski, M. Inhibition of STAT3 signal transduction and the treatment of cancer in humans. PCT Int. Appl. (2000), 92 pp. CODEN: PIXXD2 WO 2000044774 A2 20000803
Turkson, J.; Kim, J. S.; Zhang, S.; Yuan, J.; Huang, M.; Glenn, M.; Haura, E.; Sebti, S.; Hamilton, A. D.; Jove, R. Novel peptidomimetic inhibitors of signal transducer and activator of transcription 3 dimerization and biological activity Mol. Cancer Ther. 2004; 3, 261-269
Nasir, M. S.; Jolley, M. E. Fluorescence polarization: an analytical tool for immunoassay and drug discovery. Comb. Chem. High T. Scr. 1999, 2, 177-190.
Owicki, J. C. Fluorescence polarization and anisotropy in high throughput screening: perspectives and primer. J. Biomol. Screen. 2000, 5, 297-306
Shahripour, A.; Plummer, M. S.; Lunney, E.; Para, K. S.; Stankovic, C. J.; Rubin, J. R.; Humblet, C.; Fergus, J. H.; Marks, J. S.; Herrera, R.; Hubbell, S. E.; Saltiel, A. R.; Sawyer, T. K. Novel phosphotyrosine mimetics in the design of peptide ligands for pp60src SH2 domain. Bioorg. Med. Chem. Left 1996, 6, 1209-1214.
McKinney, J.; Raimundo, B. C.; Cushing, T. D.; Yoshimura, H.; Ohuchi, Y.; Hiratate, A.; Fukushima, H. Preparation of peptides as inhibitors of STAT function. U.S. (2002), 31 pp. CODEN: USXXAM US 6426331 B1 20020730.
Mckinney, J.; Raimundo, B. C.; Cushing, T. D.; Yoshimura, H.; Ohuchi, Y.; Hiratate, A.; Fukushima, H.; Xu, F.; Peto, C. STAT4 and STAT6 binding dipeptide derivatives. PCT Int. Appl. (2001), 85 pp. CODEN: PIXXD2 WO 2001083517 A1 20011108
Burke, T. R. Jr; Smyth. M. S.; Otaka, A.; Nomizu, M.; Roller, P. P; Wolf, G.; Case, R.; Shoelson, S. E Nonhydrolyzable phosphotyrosyl mimetics for the preparation of phosphatase-resistant SH2 domain inhibitors Biochemistry 1994, 33, 6490-4.
Vu, C. B.; Corpuz, E. G.; Pradeepan, S. G.; Violette, S.; Bartlett, C.; Sawyer, T. K. Nonpeptidic SH2 inhibitors of the tyrosine kinase ZAP-70. Bioorg. Med. Chem. Lett, 1999, 9, 3009-3014.
Gillespie, P.; Cicariello, J.; Olson, G. L. Conformational analysis of dipeptide mimetics Biopolymers, 1997, 43, 191-217
Amblard, M.; Daffix, I.; Berge, G.; Calmes, M.; Dodey, P.e; Pruneau, D.; Paquet, J.-L.; Luccarini, J. M.; Belichard, P.; Martinez, J. Synthesis and characterization of bradykinin B2 receptor agonists containing constrained dipeptide mimics. J. Med. Chem. 1999, 42, 4193-4201.
Arshady, R., Atherton, E., Clive, D. L. J., and Sheppard, R. C. Peptide synthesis. Part 1. Preparation and use of polar supports based on polydimethylacrylamide J. Chem. Soc. Perkin Trans. 1. 1981, 529-537.
Ren, Z.; Schaefer, T. S. ErbB-2 Activates Stat30c in a Src- and JAK2-dependent Manner J. Biol. Chem. 2002, 277, 38486-38493.
Andrews, N. C.; Falter, D. V. A rapid micropreparation technique for extraction of DNA-binding proteins from limiting numbers of mammalian cells Nuci. Acids. Res. 1991 19: 2499

1. A composition comprising a Stat3 inhibitor compound, wherein the compound comprises a structural analog of Ac-pTyr-Leu-Pro-Gln-Thr-NH₂ in which one or more amino acids have been replaced with a structural analog, wherein the compound binds to the SH2 domain of Stat3 under physiological conditions and wherein the binding of the compound inhibits a cellular signaling activity of Stat3.

2. The composition of claim 1, wherein Ac-pTyr has been replaced with 4-phosphonodifluoromethylcinnamide (F2PmCinn).

3. The composition of claim 2, wherein pivaloyloxymethyl is added to one or more of the phosphonyl oxygen atoms of 4-phosphonodifluoromethylcinnamide.

4. The composition of claim 1, wherein Ac-pTyr has been replaced with 3-phosphoryloxyindole-2-carboxylate.

5. The composition of claim 1, wherein Ac-pTyr has been replaced with 4-phosphoryloxycinnamide.

6. The composition of claim 1, wherein Ac-pTyr has been replaced with 3-phosphonodifluoromethylindole-2-carboxylate.

7. The composition of claim 6, wherein pivaloyloxymethyl is added to one or more of the phosphonyl oxygen atoms of 3-phosphonodifluoromethylindole-2-carboxylate.

8. The composition of claim 1, wherein the Leu has been replaced with cyclohexylalanine.

9. The composition of claim 1, wherein the Leu-Pro has been replaced with 5-(amino)-1,2,4,5,6,7-hexahydro-4-oxo-(2S,5S)-azepino[3,2,1-hi]indole-2-carboxylic acid (Haic).

10. The composition of claim 1, wherein the Leu-Pro has been replaced with (3S,6S,9S) 2-oxo-3-amino-1-azabicyclo[4.3.0]nonane-9-carboxylic acid (ABN).

11. The composition of claim 1, wherein the Pro has been replaced with 3,4-methanoproline.

12. The composition of claim 1, wherein the Gln has been replaced with pyrrolidinoacetamide.

13. The composition of claim 1, wherein the Thr-NH₂ has been replaced with a hydrophobic group.

14. The composition of claim 1, wherein the Thr-NH₂ has been replaced with a benzyl group.

15. The composition of claim 1, wherein Ac-pTyr has been replaced with 4-phosphoryloxycinnamide, and the Thr-NH₂ has been replaced with a benzyl group.

16. The composition of claim 1, wherein the Ac-pTyr has been replaced with 3-phosphonodifluoromethylindole-2-carboxylate, and the Thr-NH₂ has been replaced with a benzyl group.

17. The composition of claim 16, wherein pivaloyloxymethyl is added to one or more of the phosphonyl oxygen atoms of 3-phosphonodifluoromethylindole-2-carboxylate.

18. The composition of claim 1, wherein the Ac-pTyr has been replaced with 4-phosphonodifluoromethylcinnamide, the Leu has been replaced with cyclohexylalanine, and the Thr-NH₂ has been replaced with a benzyl group.

19. The composition of claim 1, wherein Ac-pTyr has been replaced with 3-phosphoryloxyindole-2-carboxylate, the Leu has been replaced with cyclohexylalanine, and the Thr-NH₂ has been replaced with a benzyl group.

20. The composition of claim 1, wherein Ac-pTyr has been replaced with 3-phosphonodifluoromethylindole-2-carboxylate, the Leu has been replaced with cyclohexylalanine, and the Thr-NH₂ has been replaced with a benzyl group.

21. The composition of claim 20, wherein pivaloyloxymethyl is added to one or more of the phosphonyl oxygen atoms of 3-phosphonodifluoromethylindole-2-carboxylate.

22. The composition of claim 1, wherein Ac-pTyr has been replaced with 4-phosphoryloxycinnamide, the Leu-Pro has been replaced with 5-(amino)-1,2,4,5,6,7-hexahydro-4-oxo-(2S,5S)-azepino[3,2,1-hi]indole-2-carboxylic acid, and the Thr-NH₂ has been replaced with a benzyl group.

23. The composition of claim 1, wherein the Ac-pTyr has been replaced with 4-phosphoryloxycinnamide, the Leu-Pro has been replaced with (3S,6S,9S) 2-oxo-3-amino-1-azabicyclo[4.3.0]nonane-9-carboxylic acid, and the Thr-NH₂ has been replaced with a benzyl group.

24. The composition of claim 1, wherein the Ac-pTyr has been replaced with 4-phosphonodifluoromethylcinnamide, the Leu-Pro has been replaced with 5-(amino)-1,2,4,5,6,7-hexahydro-4-oxo-(2S,5S)-azepino[3,2,1-hi]indole-2-carboxylic acid and the Thr-NH₂ has been replaced with a benzyl group.

25. The composition of claim 1, wherein the Ac-pTyr has been replaced with 4-phosphonodifluoromethylcinnamide, the Leu-Pro has been replaced with (3S,6S,9S) 2-oxo-3-amino-1-azabicyclo[4.3.0]nonane-9-carboxylic acid and the Thr-NH₂ has been replaced with a benzyl group.

26. The composition of claim 25, wherein pivaloyloxymethyl is added to one or more of the phosphonyl oxygen atoms of 4-phosphonodifluoromethylcinnamide.

27. The composition of claim 1, wherein Ac-pTyr has been replaced with 3-phosphoryloxyindole-2-carboxylate, the Leu-Pro has been replaced with 5-(amino)-1,2,4,5,6,7-hexahydro-4-oxo-(2S,5S)-azepino[3,2,1-hi]indole-2-carboxylic acid, and the Thr-NH₂ has been replaced with a benzyl group.

28. The composition of claim 1, wherein Ac-pTyr has been replaced with 3-phosphoryloxyindole-2-carboxylate, the Leu-Pro has been replaced with (3S,6S,9S) 2-oxo-3-amino-1-azabicyclo[4.3.0]nonane-9-carboxylic acid, and the Thr-NH₂ has been replaced with a benzyl group.

29. The composition of claim 1, wherein Ac-pTyr has been replaced with 4-phosphoryloxycinnamide, the Leu has been replaced with cyclohexylalanine, the Pro has been replaced with 3,4-methanoproline, and the Thr-NH₂ has been replaced with a benzyl group.

30. The composition of claim 1, wherein Ac-pTyr has been replaced with 4-phosphonodifluoromethylcinnamide, the Leu has been replaced with cyclohexylalanine, the Pro has been replaced with 3,4-methanoproline, and the Thr-NH₂ has been replaced with a benzyl group.

31. The composition of claim 1, wherein Ac-pTyr has been replaced with 3-phosphonodifluoromethylindole-2-carboxylate, the Leu has been replaced with cyclohexylalanine, the Pro has been replaced with 3,4-methanoproline, and the Thr-NH₂ has been replaced with a benzyl group.

32. The composition of claim 31, wherein pivaloyloxymethyl is added to one or more of the phosphonyl oxygen atoms of 3-phosphonodifluoromethylindole-2-carboxylate.

33. The composition of claim 1, wherein Ac-pTyr has been replaced with 3-phosphoryloxyindole-2-carboxylate, the Leu has been replaced with cyclohexylalanine, the Pro has been replaced with 3,4-methanoproline, and the Thr-NH₂ has been replaced with a benzyl group.

34. The composition of claim 1, wherein Ac-pTyr has been replaced with 4-phosphoryloxycinnamide, the Leu-Pro has been replaced with 5-(amino)-1,2,4,5,6,7-hexahydro-4-oxo-(2S,5S)-azepino[3,2,1-hi]indole-2-carboxylic acid, and the Gln has been replaced with pyrrolidinoacetamide.

35. The composition of claim 1, wherein Ac-pTyr has been replaced with 4-phosphoryloxycinnamide, the Leu-Pro has been replaced with (3S,6S,9S) 2-oxo-3-amino-1-azabicyclo[4.3.0]nonane-9-carboxylic acid, and the Gln has been replaced with pyrrolidinoacetamide.

36. The composition of claim 1, wherein Ac-pTyr has been replaced with 4-phosphonodifluoromethylcinnamide, the Leu-Pro has been replaced with 5-(amino)-1,2,4,5,6,7-hexahydro-4-oxo-(2S,5S)-azepino[3,2,1-hi]indole-2-carboxylic acid, and the Gln has been replaced with pyrrolidinoacetamide.

37. The composition of claim 36, wherein Ac-pTyr has been replaced with 4-phosphonodifluoromethylcinnamide, wherein pivaloyloxymethyl is added to one of the phosphonyl oxygen atoms of 4-phosphonodifluoromethylcinnamide, the Leu-Pro has been replaced with 5-(amino)-1,2,4,5,6,7-hexahydro-4-oxo-(2S,5S)-azepino[3,2, 1-hi]indole-2-carboxylic acid, and the Thr-NH₂ has been replaced with a benzyl group.

38. The composition of claim 1, wherein Ac-pTyr has been replaced with 3-phosphonodifluoromethylindole-2-carboxylate, the Leu-Pro has been replaced with 5-(amino)-1,2,4,5,6,7-hexahydro-4-oxo-(2S,5S)-azepino[3,2,1-hi]indole-2-carboxylic acid, and the Gln has been replaced with pyrrolidinoacetamide.

39. The composition of claim 38, wherein Ac-pTyr has been replaced with 3-phosphonodifluoromethylindole-2-carboxylate, wherein pivaloyloxymethyl is added to one of the phosphonyl oxygen atoms of 3-phosphonodifluoromethylindole-2-carboxylate, the Leu-Pro has been replaced with 5-(amino)-1,2,4,5,6,7-hexahydro-4-oxo-(2S,5S)-azepino[3,2,1-hi]indole-2-carboxylic acid, and the Thr-NH₂ has been replaced with a benzyl group.

40. The composition of claim 1, wherein Ac-pTyr has been replaced with 4-phosphonodifluoromethylcinnamide, the Leu-Pro has been replaced with (3S,6S,9S) 2-oxo-3-amino-1-azabicyclo[4.3.0]nonane-9-carboxylic acid, and the Gln has been replaced with pyrrolidinoacetamide.

41. The composition of claim 40, wherein Ac-pTyr has been replaced with 4-phosphonodifluoromethylcinnamide, wherein pivaloyloxymethyl is added to one of the phosphonyl oxygen atoms of 4-phosphonodifluoromethylcinnamide, the Leu-Pro has been replaced with (3S,6S,9S) 2-oxo-3-amino-i-azabicyclo[4.3.0]nonane-9-carboxylic acid, and the Thr-NH₂ has been replaced with a benzyl group.

42. The composition of claim 1, wherein Ac-pTyr has been replaced with 3-phosphonodifluoromethylindole-2-carboxylate, the Leu-Pro has been replaced with (3S,6S,9S) 2-oxo-3-amino-1-azabicyclo[4.3.0]nonane-9-carboxylic acid, and the Gln has been replaced with pyrrolidinoacetamide.

43. The composition of claim 42, wherein Ac-pTyr has been replaced with 3-phosphonodifluoromethylindole-2-carboxylate, wherein pivaloyloxymethyl is added to one of the phosphonyl oxygen atoms of 3-phosphonodifluoromethylindole-2-carboxylate, the Leu-Pro has been replaced with (3S,6S,9S) 2-oxo-3-amino-1-azabicyclo[4.3.0]nonane-9-carboxylic acid, and the Thr-NH₂ has been replaced with a benzyl group.

44. The compositions of claims 1-43, wherein the compound further comprises a membrane transporter sequence.

45. The compositions of claim 44, wherein the membrane transporter sequence is Ala-Ala-Val-Leu-Leu-Pro-Val-Leu-Leu-Ala-Ala-Pro-NH₂.

46. The composition of any of claims 1-43, wherein the compound is dissolved or suspended in a pharmaceutically acceptable carrier.

47. A composition comprising a compound having the structure: F2PmCinn-Leu-Pro-Gln-Thr-Val-Ala-Ala-Val-Leu-Leu-Pro-Val-Leu-Leu-Ala- Ala-Pro-NH₂.

48. A method of inhibiting the signaling activity of Stat3 in a cell comprising contacting the cell with a compound that binds to the SH2 domain of Stat3, wherein the molecule comprises a structural analog of phosphorylated Tyr 904 of gp130.

49. The method of claim 48, wherein the compound comprises a structural analog of Ac-pTyr-Leu-Pro-Gln-Thr-NH₂ in which one or more amino acids have been replaced with a structural analog.

50. The method of claim 48, wherein the compound binds to the SH2 domain and inhibits Stat3 dimerization.

51. The method of claim 48, wherein binding of the compound to Stat3 inhibits translocation of Stat3 to the nucleus of the cell.

52. The method of claim 48, wherein binding of the compound to Stat3 inhibits activation of transcription of Stat3 responsive genes in the cell.
